(19) United States
(12) Patent Application Publication
(10) Pub. No.: US 2025/0345326 A1
(43) Pub. Date: Nov. 13, 2025

(54) METHODS FOR TREATING CHOLESTATIC PRURITUS

(71) Applicant: Mirum Pharmaceuticals, Inc, Foster City, CA (US)

(72) Inventors: Pamela VIG, Foster City, CA (US); Douglas B. MOGUL, Foster City, CA (US); Tiago NUNES, Foster City, CA (US)

(73) Assignee: Mirum Pharmaceuticals, Inc, Foster City, CA (US)

(21) Appl. No.: 19/200,443

(22) Filed: May 6, 2025

Related U.S. Application Data
(60) Provisional application No. 63/643,524, filed on May 7, 2024.
(60) Provisional application No. 63/646,271, filed on May 13, 2024.
(60) Provisional application No. 63/673,284, filed on Jul. 19, 2024.

(51) Int. Cl. A61K 31/4995; A61K 9/00

(52) U.S. Cl. CPC A61K 31/4995; A61K 9/0053

(57) ABSTRACT

Provided herein are methods for treating cholestatic pruritus in a subject having a cholestatic liver disease. The method includes administering maralixibat to the subject. More specifically, the present invention relates to methods for treating cholestatic pruritus in a subject having a rare cholestatic liver disease wherein the method comprises administering maralixibat to the subject at a dose of at least 300 μg/kg/day.

### CROSS REFERENCE TO RELATED APPLICATIONS

This application claims the benefit under 35 U.S.C. § 119 (e) of U.S. Provisional Application Nos. 63/643,524, filed May 7, 2024, 63/646,271, filed May 13, 2024, and 63/673,284, filed Jul. 19, 2024, which are incorporated herein by reference in their entirety for all purposes.

### FIELD OF INVENTION

The present invention relates generally to methods for treating cholestatic pruritus in a subject. More specifically, the present invention relates to methods for treating cholestatic pruritus in a subject having a rare disease where the method comprises administering maralixibat to the subject at a dose of at least 300 μg/kg/day.

### BACKGROUND OF THE INVENTION

Cholestatic pruritus significantly impacts patients across a wide range of acute and chronic liver diseases. However, the prevalence of pruritus burden is only known for the most common cholestatic liver diseases, with no data available for very rare indications. In primary biliary cholangitis (PBC), the most common chronic cholestatic liver disease (prevalence of 29 per 100,000 persons), pruritus has been reported in up to 80% of patients (Kremer et al. 2011; Lu et al. 2018). Cholestatic pruritus predominates in patients with progressive familial intrahepatic cholestasis (PFIC), a disease with a prevalence of 1 in 50,000 to 100,000 persons (Davit-Spraul et al. 2009). In Alagille syndrome (ALGS), with a prevalence of 1 in 30,000 persons, pruritus has been reported in up to 75% of patients (Baker et al. 2019; Vandriel et al. 2022). In addition to being a frequent complication for these prototypic cholestatic liver diseases, pruritus has been described in several other rare and ultrarare diseases. Patients with primary sclerosing cholangitis (PSC) and other disorders of intrahepatic and extrahepatic biliary transport such as biliary atresia (BA), IgG4 sclerosing cholangitis, and intrahepatic cholestasis of pregnancy (ICP) also may experience pruritus as part of their liver disease (Beuers et al. 2023).

In addition to the prevalence of pruritus, this symptom is often reported as among the most burdensome complication in people with cholestatic liver diseases. Pruritus often leads to sleep disturbances, fatigue, irritability, cutaneous self-mutilation, poor attention and school performance, and impaired quality of life (QoL). For example, in PFIC, pruritus is reported to be severe in 76%-80% of patients and generally regarded as the most bothersome symptom such that surgical intervention through biliary diversion or liver transplantation is often necessary (Whitington et al. 1994; Lee et al. 2009; Baker et al. 2019). In ALGS, cholestatic pruritus is reported by patients and caregivers as the most problematic symptom of ALGS across all ages (Kamath et al. 2018), leading to disruption of social and educational activities and negatively impacting psychosocial health (Elisofon et al. 2010; Kamath et al. 2015, 2018) and overall QoL (Abetz-Webb et al. 2014); pruritis is a key indication for liver transplant in more than two-thirds of patients with ALGS. Cholestatic pruritus is a distressing symptom of PBC that can impact the QoL of patients and their daily activities, including sleep, resulting in fatigue and depression (Lindor et al. 2009). In PSC, pruritus affects up to 70% of patients and leads to impaired QoL (Haapamaki et al. 2015; Raszeja-Wyszomirska et al. 2015; Cheung et al. 2016; PSC Support 2016; Kuo et al. 2019). Pruritus in PSC is thought to be underreported and undertreated based on findings in a recent observational study (Kuo et al. 2019).

In rarer cholestatic liver diseases, such as the ones being discussed presently, the specific prevalence and severity of pruritus is not yet known. For example, in biliary atresia (BA), pruritus may sometimes present later on and prior to transplantation. BA is a rare, inflammatory condition of the biliary tree that presents in the first weeks of life and involves extrahepatic bile duct obstruction. This is associated with consequent liver injury, fibrosis, and cirrhosis that leads to portal hypertension and a decline in hepatic synthetic function. Kasai hepatoportoenterostomy (Kasai HPE or Kasai) is the standard of care as first-line intervention, generally being performed within the first 2 months of life. Although HPE frequently improves the signs and symptoms of BA, a substantial proportion of patients do not respond fully. Some patients with BA who have had HPE still develop cholestatic pruritus that is significant enough to greatly impair QoL for both patient and family (Shneider and Mazariegos 2007, Hirschfield 2011, Sundaram et al. 2017). At present, there are no approved pharmacological agents that have demonstrated efficacy in reducing pruritus in patients with post-HPE BA, and available medical approaches have limited efficacy.

There are currently no approved drugs for the treatment of cholestatic pruritus in liver diseases other than ALGS and PFIC, as other indications are often too rare to feasibly conduct clinical studies. Off-label agents (e.g., cholestyramine, rifampicin, and naltrexone) are generally used for the management of cholestatic pruritus with typically limited success and safety or tolerability concerns (Kronsten et al. 2013).

Thus, there exists a significant unmet need for treatment of cholestatic pruritus in rare diseases, especially those diseases where individual clinical studies cannot be conducted and those where there is currently no approved therapy.

### SUMMARY OF THE INVENTION

Various non-limiting aspects and embodiments of the invention are described below.

In one aspect, the present disclosure provides a method for treating a rare cholestatic liver disease in a subject in need thereof comprising administering to the subject about 300 μg/kg/day to about 1200 μg/kg/day maralixibat, or a pharmaceutically acceptable salt thereof.

In another aspect, the present disclosure provides a method of reducing cholestatic pruritus in a subject having a rare cholestatic liver disease comprising administering to the subject about 300 μg/kg/day to about 1200 μg/kg/day maralixibat, or a pharmaceutically acceptable salt thereof.

In one embodiment, the rare cholestatic liver disease is selected from the group consisting of Caroli disease, Caroli syndrome, ciliopathies (Joubert syndrome, Meckel-Gruber syndrome, NPHP3 mutation), alpha-1-antitrypsin deficiency, chronic idiopathic hepatitis, secondary sclerosing cholangitis related to COVID-19, secondary sclerosing cholangitis related to injury, IgG4-related sclerosing cholangitis, ARC, Langerhans cell histiocytosis, sodium taurocholate co-transporting polypeptide deficiency, transaldolase deficiency, X-linked myotubular myopathy, hepatic sarcoidosis, idiopathic amyloidosis, ischemic cholangiopathy, rare metabolic disorders, post-liver transplant cholestasis, and undiagnosed cholestatic pruritus. In one embodiment, the rare cholestatic liver disease is not Alagille syndrome (ALGS), progressive familial intrahepatic cholestasis (PFIC), primary biliary cholangitis (PBC), or primary sclerosing cholangitis (PSC).

In one embodiment, the rare cholestatic liver disease is biliary atresia (BA). In one embodiment, the rare cholestatic liver disease is post-liver transplant cholestasis.

In one embodiment, the subject with post-liver transplant cholestasis has Alagille syndrome (ALGS), progressive familial intrahepatic cholestasis (PFIC), primary biliary cholangitis (PBC), or primary sclerosing cholangitis (PSC).

In one embodiment, the subject has a rare metabolic disorder selected from the group consisting of an organic acidemia, an amino acid disorder, a disorder of glucose metabolism, a congenital disorder of glycosylation (CDG), and a mitochondrial disorder. In one embodiment, the organic acidemias comprise, but are not limited to, methylmalonic acidemia and propionic acidemia. In one embodiment, the amino acid disorders comprise, but are not limited to, maple syrup urine disease and tyrosinemia. In one embodiment, the disorders of glucose metabolism comprise, but are not limited to, glycogen storage disease, galactosemia, and transaldolase deficiency. In one embodiment, the congenital disorders of glycosylation (CDGs) comprise, but are not limited to, disorders of protein N-glycosylation, disorders of protein O-glycosylation, disorders of glycosphingolipid and GPI-anchor glycosylation, and defects of multiple glycosylation. In one embodiment, the mitochondrial disorders include, but are not limited to, mitochondrial encephalomyopathy with lactic acidosis and stroke-like episodes (MELAS), mitochondrial neurogastrointestinal encephalomyopathy (MNGIE), and Pearson syndrome.

In one embodiment, the treatment comprises reducing one or more of cholestatic pruritus, serum bile acids, and bilirubin. In one embodiment, the treatment comprises reducing cholestatic pruritus.

In one embodiment, the pharmaceutically acceptable salt of maralixibat is maralixibat chloride, maralixibat bromide, maralixibat acetate, or maralixibat mesylate. In one embodiment, the pharmaceutically acceptable salt of maralixibat is maralixibat chloride.

In one embodiment, the maralixibat or pharmaceutically acceptable salt thereof is administered in an amount from about 400 μg/kg/day to about 1200 μg/kg/day.

In one embodiment, the maralixibat or pharmaceutically acceptable salt thereof is administered in an amount from about 600 μg/kg/day to about 1200 μg/kg/day. In one embodiment, the maralixibat or pharmaceutically acceptable salt thereof is maralixibat chloride, and maralixibat chloride is administered in an amount of about 1200 μg/kg/day.

In one embodiment, the maralixibat or pharmaceutically acceptable salt thereof is administered in an amount of about 0.5 mg/day to about 100 mg/day. In one embodiment, the maralixibat or pharmaceutically acceptable salt thereof is administered in an amount of about 10 mg/day to about 60 mg/day.

In one embodiment, the subject has intermittent cholestasis. In one embodiment, the subject has undergone biliary diversion surgery. In one embodiment, the subject has undergone liver transplantation.

In one embodiment, the subject is a pediatric subject. In one embodiment, the subject is more than 1 year old and less than 18 years old. In one embodiment, the subject is less than 12 months old.

In one embodiment, the subject is an adult.

In one embodiment, the maralixibat or pharmaceutically acceptable salt thereof is administered once daily (QD). In one embodiment, the maralixibat or pharmaceutically acceptable salt thereof is administered twice daily (BID).

In one embodiment, the maralixibat or pharmaceutically acceptable salt thereof is maralixibat chloride, and maralixibat chloride is administered at 600 μg/kg/day BID for a total daily dose of 1200 μg/kg/day.

In one embodiment, the reduction of cholestatic pruritus is a reduction of an ItchRO score, of an ItchRO(Obs) score, of a CSS score, of a patient impression of severity of pruritus (PIS), of a caregiver impression of severity of pruritus (CIS), of a patient impression of change (PIC), or of a caregiver impression of change (CIC), or a combination thereof.

In one embodiment, the administration of the maralixibat or pharmaceutically acceptable salt thereof reduces an ItchRO(Obs) score of the subject by at least 1.0 points relative to baseline. In one embodiment, the administration of the maralixibat or pharmaceutically acceptable salt thereof reduces an ItchRO(Obs) score of the subject by at least 1.2 points relative to baseline. In one embodiment, the administration of the maralixibat or pharmaceutically acceptable salt thereof reduces an ItchRO(Obs) score of the subject by at least 1.4 points relative to baseline. In one embodiment, the administration of the maralixibat or pharmaceutically acceptable salt thereof reduces an ItchRO(Obs) score of the subject by at least 1.6 points relative to baseline.

In one embodiment, the administration of the maralixibat or pharmaceutically acceptable salt thereof reduces a CSS score of the subject by at least 1.0 points relative to baseline. In one embodiment, the administration of the maralixibat or pharmaceutically acceptable salt thereof reduces a CSS score of the subject by at least 1.2 points relative to baseline. In one embodiment, the administration of the maralixibat or pharmaceutically acceptable salt thereof reduces a CSS score of the subject by at least 1.4 points relative to baseline. In one embodiment, the administration of the maralixibat or pharmaceutically acceptable salt thereof reduces a CSS score of the subject by at least 1.6 points relative to baseline.

In one embodiment, the administration of the maralixibat or pharmaceutically acceptable salt thereof reduces sBA concentration in the subject by at least 50 μmol/L relative to baseline.

In one embodiment, the administration of the maralixibat or pharmaceutically acceptable salt thereof reduces total bilirubin by at least 0.2 mg/dL relative to baseline.

In one embodiment, any of the above methods further comprises administering a lipid soluble vitamin (LSV) in subjects with LSV deficiency. In one embodiment, the LSV is selected from the group consisting of Vitamin A, Vitamin D and Vitamin E.

In one embodiment, the maralixibat or pharmaceutically acceptable salt thereof is administered before a meal. In one embodiment, the maralixibat or pharmaceutically acceptable salt thereof is administered about 30 minutes before a meal.

In one embodiment, the maralixibat or pharmaceutically acceptable salt thereof is administered BID, about 30 minutes before the morning meal and about 30 minutes before the evening meal.

In one embodiment, the maralixibat is administered in the form of a pharmaceutical composition comprising maralixibat, or a pharmaceutically acceptable salt thereof, an antioxidant, and a preservative. In one embodiment, the pharmaceutical composition is a liquid composition for oral administration. In one embodiment, the liquid composition is an aqueous solution.

In one embodiment, the maralixibat is present in an amount of about 2 mg/mL to about 100 mg/mL of the composition. In one embodiment, the maralixibat is present in an amount of about 5 mg/mL to about 50 mg/mL of the composition. In one embodiment, the maralixibat is present in an amount of about 8 mg/mL to about 20 mg/mL of the composition.

In one embodiment, the pharmaceutical composition is a solid composition for oral administration.

These and other aspects of the present invention will become apparent to those skilled in the art after a reading of the following detailed description of the invention, including the appended claims.

### BRIEF DESCRIPTION OF THE DRAWINGS

FIG. 1 is a study schema of the presently described clinical study.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Bile acids/salts play a critical role in activating digestive enzymes and solubilizing fats and fat-soluble vitamins and are involved in liver, biliary, and intestinal disease. Bile acids are synthesized in the liver by a multistep, multiorganelle pathway. Hydroxyl groups are added to specific sites on the steroid structure, the double bond of the cholesterol B ring is reduced, and the hydrocarbon chain is shortened by three carbon atoms resulting in a carboxyl group at the end of the chain. The most common bile acids are cholic acid and chenodeoxycholic acid (the “primary bile acids”). Before exiting the hepatocytes and forming bile, the bile acids are conjugated to either glycine (to produce glycocholic acid or glycochenodeoxycholic acid) or taurine (to produce taurocholic acid or taurochenodeoxycholic acid). The conjugated bile acids are called bile salts and their amphipathic nature makes them more efficient detergents than bile acids. Bile salts, not bile acids, are found in bile.

Bile salts are excreted by the hepatocytes into the canaliculi to form bile. The canaliculi drain into the right and left hepatic ducts and the bile flows to the gallbladder. Bile is released from the gallbladder and travels to the duodenum, where it contributes to the metabolism and degradation of fat. The bile salts are reabsorbed in the terminal ileum and transported back to the liver via the portal vein. Bile salts often undergo multiple enterohepatic circulations before being excreted via feces. A small percentage of bile salts may be reabsorbed in the proximal intestine by either passive or carrier-mediated transport processes. Most bile salts are reclaimed in the distal ileum by a sodium-dependent apically located bile acid transporter referred to as apical sodium-dependent bile acid transporter (ASBT). At the basolateral surface of the enterocyte, a truncated version of ASBT is involved in vectoral transfer of bile acids/salts into the portal circulation. Completion of the enterohepatic circulation occurs at the basolateral surface of the hepatocyte by a transport process that is primarily mediated by a sodium-dependent bile acid transporter. Intestinal bile ac-id transport plays a key role in the enterohepatic circulation of bile salts. Molecular analysis of this process has recently led to important advances in understanding of the biology, physiology and pathophysiology of intestinal bile acid transport.

Within the intestinal lumen, bile acid concentrations vary, with the bulk of the reuptake occurring in the distal intestine. Described herein are certain compositions and methods that control bile acid concentrations in the intestinal lumen, thereby controlling the hepatocellular damage caused by bile acid accumulation in the liver.

### Classes of Cholestasis and Cholestatic Liver Disease

As used herein, “cholestasis” means the disease or symptoms comprising impairment of bile formation and/or bile flow. As used herein, “cholestatic liver disease” means a liver disease associated with cholestasis. Cholestatic liver diseases are often associated with jaundice, fatigue, and pruritis. Biomarkers of cholestatic liver disease include elevated serum bile acid concentrations, elevated serum alkaline phosphatase (AP), elevated gamma-glutamyltranspeptidease, elevated conjugated hyperbilirubinemia, and elevated serum cholesterol.

Cholestatic liver disease can be sorted clinicopathologically between two principal categories of obstructive, often extrahepatic, cholestasis, and nonobstructive, or intrahepatic, cholestasis. In the former, cholestasis results when bile flow is mechanically blocked, as by gallstones or tumor, or as in extrahepatic biliary atresia.

The latter group who has nonobstructive intrahepatic cholestasis in turn fall into two principal subgroups. In the first subgroup, cholestasis results when processes of bile secretion and modification, or of synthesis of constituents of bile, are caught up secondarily in hepatocellular injury so severe that nonspecific impairment of many functions can be expected, including those subserving bile formation. In the second subgroup, no presumed cause of hepatocellular injury can be identified. Cholestasis in such patients appears to result when one of the steps in bile secretion or modification, or of synthesis of constituents of bile, is constitutively damaged. Such cholestasis is considered primary.

Accordingly, provided herein are methods and compositions for stimulating epithelial proliferation and/or regeneration of intestinal lining and/or enhancement of the adaptive processes in the intestine in individuals with hypercholemia and/or a cholestatic liver disease. In some of such embodiments, the methods comprise increasing bile acid concentrations and/or GLP-2 concentrations in the intestinal lumen.

Increased levels of bile acids, and elevated levels of AP (alkaline phosphatase), LAP (leukocyte alkaline phosphatase), gamma GT (gamma-glutamyl transpeptidase), and 5′-nucleotidase are bio-chemical hallmarks of cholestasis and cholestatic liver disease. Accordingly, provided herein are methods and compositions for stimulating epithelial proliferation and/or regeneration of intestinal lining and/or enhancement of the adaptive processes in the intestine in individuals with hypercholemia, and elevated levels of AP (alkaline phosphatase), LAP (leukocyte alkaline phosphatase), gamma GT (gamma-glutamyl transpeptidase or GGT), and/or 5′-nucleotidase. In some of such embodiments, the methods comprise increasing bile acid concentrations in the intestinal lumen. Further provided herein, are methods and compositions for reducing hypercholemia, and elevated levels of AP (alkaline phosphatase), LAP (leukocyte alkaline phosphatase), gamma GT (gamma-glutamyl transpeptidase), and 5′-nucleotidase comprising reducing overall serum bile acid load by excreting bile acid in the feces.

Pruritus is often associated with hypercholemia and cholestatic liver diseases. Pruritus associated with cholestatic liver diseases is known as cholestatic pruritus. It has been suggested that cholestatic pruritus results from bile salts acting on peripheral pain afferent nerves. The degree of cholestatic pruritus varies with the individual (i.e., some individuals are more sensitive to elevated levels of bile acids/salts).

Administration of agents that reduce serum bile acid concentrations has been shown to reduce cholestatic pruritus in certain individuals. Accordingly, provided herein are methods and compositions for stimulating epithelial proliferation and/or regeneration of intestinal lining and/or enhancement of the adaptive processes in the intestine in individuals with cholestatic pruritus. In some of such embodiments, the methods comprise increasing bile acid concentrations in the intestinal lumen. Further provided herein, are methods and compositions for treating cholestatic pruritus comprising reducing overall serum bile acid load by excreting bile acid in the feces.

Another symptom of hypercholemia and cholestatic liver disease is the increase in serum concentration of conjugated bilirubin. Elevated serum concentrations of conjugated bilirubin result in jaundice and dark urine. The magnitude of elevation is not diagnostically important as no relationship has been established between serum levels of conjugated bilirubin and the severity of hypercholemia and cholestatic liver disease. Conjugated bilirubin concentration rarely exceeds 30 mg/dL. Accordingly, provided herein are methods and compositions for stimulating epithelial proliferation and/or regeneration of intestinal lining and/or enhancement of the adaptive processes in the intestine in individuals with elevated serum concentrations of conjugated bilirubin. In some of such embodiments, the methods comprise increasing bile acid concentrations in the intestinal lumen. Further provided herein, are methods and compositions for treating elevated serum concentrations of conjugated bilirubin comprising reducing overall serum bile acid load by excreting bile acid in the feces.

Increased serum concentration of nonconjugated bilirubin is also considered diagnostic of hypercholemia and cholestatic liver disease. Portions of serum bilirubin and covalently bound to albumin (delta bilirubin or biliprotein). This fraction may account for a large proportion of total bilirubin in patients with cholestatic jaundice. The presence of large quantities of delta bilirubin indicates long-standing cholestasis. Delta bilirubin in cord blood or the blood of a newborn is indicative of cholestasis/cholestatic liver disease that antedates birth. Accordingly, provided herein are methods and compositions for stimulating epithelial proliferation and/or regeneration of intestinal lining and/or enhancement of the adaptive processes in the intestine in individuals with elevated serum concentrations of nonconjugated bilirubin or delta bilirubin. In some of such embodiments, the methods comprise increasing bile acid concentrations in the intestinal lumen. Further provided herein, are methods and compositions for treating elevated serum concentrations of nonconjugated bilirubin and delta bilirubin comprising reducing overall serum bile acid load by excreting bile acid in the feces.

Cholestasis and cholestatic liver disease results in hypercholemia. During metabolic cholestasis, the hepatocytes retains bile salts. Bile salts are regurgitated from the hepatocyte into the serum, which results in an increase in the concentration of bile salts in the peripheral circulation. Furthermore, the uptake of bile salts entering the liver in portal vein blood is inefficient, which results in spillage of bile salts into the peripheral circulation. Accordingly, provided herein are methods and compositions for stimulating epithelial proliferation and/or regeneration of intestinal lining and/or enhancement of the adaptive processes in the intestine in individuals with hypercholemia. In some of such embodiments, the methods comprise increasing bile acid concentrations in the intestinal lumen. Further provided herein, are methods and compositions for treating hypercholemia comprising reducing overall serum bile acid load by excreting bile acid in the feces.

Hyperlipidemia is characteristic of some but not all cholestatic diseases. Serum cholesterol is elevated in cholestasis due to the decrease in circulating bile salts which contribute to the metabolism and degradation of cholesterol. Cholesterol retention is associated with an increase in mem-brane cholesterol content and a reduction in membrane fluidity and membrane function. Furthermore, as bile salts are the metabolic products of cholesterol, the reduction in cholesterol metabolism results in a decrease in bile acid/salt synthesis. Serum cholesterol observed in children with cholestasis ranges between about 1,000 mg/dL and about 4,000 mg/dL. Accordingly, pro-vided herein are methods and compositions for stimulating epithelial proliferation and/or regeneration of intestinal lining and/or enhancement of the adaptive processes in the intestine in individuals with hyperlipidemia. In some of such embodiments, the methods comprise increasing bile acid concentrations in the intestinal lumen. Further provided herein, are methods and compositions for treating hyperlipidemia comprising reducing overall serum bile acid load by excreting bile acid in the feces.

In individuals with hypercholemia and cholestatic liver diseases, xanthomas develop from the deposition of excess circulating cholesterol into the dermis. The development of xanthomas is more characteristic of obstructive cholestasis than of hepatocellular cholestasis. Planar xanthomas first occur around the eyes and then in the creases of the palms and soles, followed by the neck. Tuberous xanthomas are associated with chronic and long-term cholestasis. Accordingly, provided herein are methods and compositions for stimulating epithelial proliferation and/or regeneration of intestinal lining and/or enhancement of the adaptive processes in the intestine in individuals with xanthomas. In some of such embodiments, the methods comprise increasing bile acid concentrations in the intestinal lumen. Further provided herein, are methods and compositions for treating xanthomas comprising reducing overall serum bile acid load by excreting bile acid in the feces.

In children with chronic cholestasis, one of the major consequences of hypercholemia and cholestatic liver disease is failure to thrive. Failure to thrive is a consequence of reduced delivery of bile salts to the intestine, which contributes to inefficient digestion and absorption of fats, and reduced uptake of vitamins (vitamins E, D, K, and A are all malabsorbed in cholestasis). Further-more, the delivery of fat into the colon can result in colonic secretion and diarrhea. Treatment of failure to thrive involves dietary substitution and supplementation with long-chain triglycerides, medium-chain triglycerides, and vitamins. Ursodeoxycholic acid, which is used to treat some cholestatic conditions, does not form mixed micelles and has no effect on fat absorption. Accordingly, provided herein are methods and compositions for stimulating epithelial proliferation and/or regeneration of intestinal lining and/or enhancement of the adaptive processes in the intestine in individuals (e.g., children) with failure to thrive. In some of such embodiments, the methods comprise increasing bile acid concentrations in the intestinal lumen. Further provided herein, are methods and compositions for treating failure to thrive comprising reducing overall serum bile acid load by excreting bile acid in the feces.

Cholestatic pruritus is increasingly recognized as being largely driven by elevated serum bile acids (sBAs), irrespective of the specific etiology of liver disease (Thébaut et al. 2018; Patel et al. 2019; Yu et al. 2021). Clinical studies affirm improvements in cholestatic pruritus following use of IBAT inhibitors (i.e., maralixibat (Livmarli®) and odevixibat (Bylvay®)), a class of drugs with a targeted mechanism of action that blocks reuptake of bile acids in the distal small bowel. In Alagille syndrome (ALGS), a disease characterized by paucity of intrahepatic bile ducts that leads to severe pruritus, and progressive familial intrahepatic cholestasis (PFIC), a heterogeneous group of different genetic disorders characterized by abnormalities in bile formation, IBAT inhibitors are approved treatments based on evidence of rapid improvements in pruritus and corresponding reductions in sBAs. In PFIC, regardless of the underlying mechanism for each type, patients experience cholestatic pruritus, which is responsive to IBAT inhibitors. Lastly, IBAT inhibitors have been evaluated with promising results for the treatment of cholestatic pruritus in primary biliary cholangitis (PBC) and primary sclerosing cholangitis (PSC), two immune-mediated diseases that affect bile ducts and lead to chronic cholestasis.

Maralixibat inhibits IBAT in the terminal ileum, increasing bile acid excretion that results in reduced sBA levels. These net reductions in sBA have been shown to have an efficacious impact on cholestatic pruritus in patients with cholestatic liver disease such as ALGS and PFIC. It is hypothesized that improvements in pruritus by interruption of the enterohepatic circulation with IBAT inhibition may also be seen in other cholestatic liver diseases in which there are elevated bile acids and pruritus.

Maralixibat is known as Livmarli®, SHP625, LUM001, lopixibat, or 1-[[4-[[4-[(4R,5R)-3,3-dibutyl-7-(dimethylamino)-2,3,4,5-tetrahydro-4-hydroxy-1,1-dioxido-1-benzothiepin-5-yl]phenoxy]methyl]phenyl]methyl]-4-aza-1-azoniabicyclo[2.2.2]octane. The structure of maralixibat in the free base form is shown below:

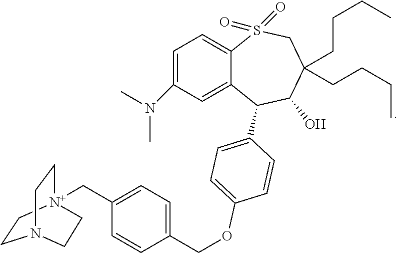 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O]

The structure of maralixibat chloride is shown below:

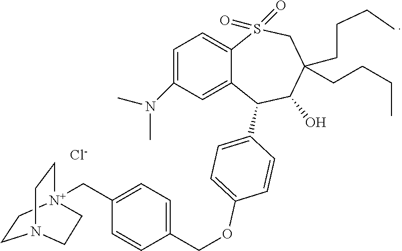 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present embodiments include pharmaceutically acceptable salt of the compounds described herein. The present embodiments also include quaternary ammonium salts of the compounds described herein, where the compounds have one or more tertiary amine moieties. In one embodiment, the pharmaceutically acceptable salt of maralixibat is maralixibat chloride.

The IBAT is ideally suited for pharmacological modulation of bile acid transport by a compound that can be restricted to the lumen of the gut, one that is not required to have systemic exposure for activity. Maralixibat was designed to be minimally absorbed due to its large molecular weight (˜710 Da) and the presence of a positively charged quaternary nitrogen atom, therefore maximizing the local exposure of the molecule to its target and minimizing unnecessary systemic exposure.

Because reductions in sBA concentrations after surgical interruption of the enterohepatic circulation have been shown to be associated with improvements in cholestasis and clinical outcomes in several pediatric cholestatic liver diseases pharmacological interruption of the enterohepatic circulation by IBAT inhibition represents a potential nonsurgical and easily reversible alternative to achieve a similar reduction in sBA, and thus has the potential to improve outcomes in diseases such as PFIC, ALGS, and biliary atresia.

### Bile Acid

Bile contains water, electrolytes and a numerous organic molecules including bile acids, cholesterol, phospholipids and bilirubin. Bile is secreted from the liver and stored in the gall bladder, and upon gall bladder contraction, due to ingestion of a fatty meal, bile passes through the bile duct into the intestine. Bile acids/salts are critical for digestion and absorption of fats and fat-soluble vitamins in the small intestine. Adult humans produce 400 to 800 mL of bile daily. The secretion of bile can be considered to occur in two stages. Initially, hepatocytes secrete bile into canaliculi, from which it flows into bile ducts and this hepatic bile contains large quantities of bile acids, cholesterol and other organic molecules. Then, as bile flows through the bile ducts, it is modified by addition of a watery, bicarbonate-rich secretion from ductal epithelial cells. Bile is concentrated, typically five-fold, during storage in the gall bladder.

The flow of bile is lowest during fasting, and a majority of that is diverted into the gallbladder for concentration. When chyme from an ingested meal enters the small intestine, acid and partially digested fats and proteins stimulate secretion of cholecystokinin and secretin, both of which are important for secretion and flow of bile. Cholecystokinin (cholecysto=gallbladder and kinin=movement) is a hormone which stimulates contractions of the gallbladder and common bile duct, resulting in delivery of bile into the gut. The most potent stimulus for release of cholecystokinin is the presence of fat in the duodenum. Secretin is a hormone secreted in response to acid in the duodenum, and it simulates biliary duct cells to secrete bicarbonate and water, which expands the volume of bile and increases its flow out into the intestine.

Bile acids/salts are derivatives of cholesterol. Cholesterol, ingested as part of the diet or derived from hepatic synthesis, are converted into bile acids/salts in the hepatocyte. Examples of such bile acids/salts include cholic and chenodeoxycholic acids, which are then conjugated to an amino acid (such as glycine or taurine) to yield the conjugated form that is actively secreted into canaliculi. The most abundant of the bile salts in humans are cholate and deoxycholate, and they are normally conjugated with either glycine or taurine to give glycocholate or taurocholate respectively.

Free cholesterol is virtually insoluble in aqueous solutions, however in bile it is made soluble by the presence of bile acids/salts and lipids. Hepatic synthesis of bile acids/salts accounts for the majority of cholesterol breakdown in the body. In humans, roughly 500 mg of cholesterol are converted to bile acids/salts and eliminated in bile every day. Therefore, secretion into bile is a major route for elimination of cholesterol. Large amounts of bile acids/salts are secreted into the intestine every day, but only relatively small quantities are lost from the body. This is because approximately 95% of the bile acids/salts delivered to the duodenum are absorbed back into blood within the ileum, by a process is known as “Enterohepatic Recirculation”.

Venous blood from the ileum goes straight into the portal vein, and hence through the sinusoids of the liver. Hepatocytes extract bile acids/salts very efficiently from sinusoidal blood, and little escapes the healthy liver into systemic circulation. Bile acids/salts are then transported across the hepatocytes to be resecreted into canaliculi. The net effect of this enterohepatic recirculation is that each bile salt molecule is reused about 20 times, often two or three times during a single digestive phase. Bile biosynthesis represents the major metabolic fate of cholesterol, accounting for more than half of the approximate 800 mg/day of cholesterol that an average adult uses up in metabolic processes. In comparison, steroid hormone biosynthesis consumes only about 50 mg of cholesterol per day. Much more that 400 mg of bile salts is required and secreted into the intestine per day, and this is achieved by re-cycling the bile salts. Most of the bile salts secreted into the upper region of the small intestine are absorbed along with the dietary lipids that they emulsified at the lower end of the small intestine. They are separated from the dietary lipid and returned to the liver for re-use. Recycling thus enables 20-30 g of bile salts to be secreted into the small intestine each day.

Bile acids/salts are amphipathic, with the cholesterol-derived portion containing both hydrophobic (lipid soluble) and polar (hydrophilic) moieties while the amino acid conjugate is generally polar and hydrophilic. This amphipathic nature enables bile acids/salts to carry out two important functions: emulsification of lipid aggregates and solubilization and transport of lipids in an aqueous environment. Bile acids/salts have detergent action on particles of dietary fat which causes fat globules to break down or to be emulsified. Emulsification is important since it greatly increases the surface area of fat available for digestion by lipases which cannot access the inside of lipid droplets. Furthermore, bile acids/salts are lipid carriers and are able to solubilize many lipids by forming micelles and are critical for transport and absorption of the fat-soluble vitamins.

The term “non-systemic” or “minimally absorbed,” as used herein, refers to low systemic bioavailability and/or absorption of an administered compound. In some embodiments a non-systemic compound is a compound that is substantially not absorbed systemically. In some embodiments, ASBTI compositions described herein deliver the ASBTI to the distal ileum, colon, and/or rectum and not systemically (e.g., a substantial portion of the ASBTI is not systemically absorbed. In some embodiments, the systemic absorption of a non-systemic compound is <0.1%, <0.3%, <0.5%, <0.6%, <0.7%, <0.8%, <0.9%, <1%, <1.5%, <2%, <3%, or <5% of the administered dose (wt. % or mol %). In some embodiments, the systemic absorption of a non-systemic compound is <10% of the administered dose. In some embodiments, the systemic absorption of a non-systemic compound is <15% of the administered dose. In some embodiments, the systemic absorption of a non-systemic compound is <25% of the administered dose. In an alternative approach, a non-systemic ASBTI (e.g., maralixibat) is a compound that has lower systemic bioavailability relative to the systemic bioavailability of a systemic ASBTI. In some embodiments, the bioavailability of a non-systemic ASBTI described herein (e.g., maralixibat) is <30%, <40%, <50%, <60%, or <70% of the bioavailability of a systemic ASBTI.

In another alternative approach, compositions described herein are formulated to deliver <10% of the administered dose of the ASBTI systemically. In some embodiments, the compositions described herein are formulated to deliver <20% of the administered dose of the ASBTI systemically. In some embodiments, the compositions described herein are formulated to deliver <30% of the administered dose of the ASBTI systemically. In some embodiments, the compositions described herein are formulated to deliver <40% of the administered dose of the ASBTI systemically. In some embodiments, the compositions described herein are formulated to deliver <50% of the administered dose of the ASBTI systemically. In some embodiments, the compositions described herein are formulated to deliver <60% of the administered dose of the ASBTI systemically. In some embodiments, the compositions described herein are formulated to deliver <70% of the administered dose of the ASBTI systemically. In some embodiments, systemic absorption is determined in any suitable manner, including the total circulating amount, the amount cleared after administration, or the like.

Bile acids/salts play a critical role in activating digestive enzymes and solubilizing fats and fat-soluble vitamins and are involved in liver, biliary, and intestinal disease. Bile acids are synthesized in the liver by a multistep, multiorganelle pathway. Hydroxyl groups are added to specific sites on the steroid structure, the double bond of the cholesterol B ring is reduced, and the hydrocarbon chain is shortened by three carbon atoms resulting in a carboxyl group at the end of the chain. The most common bile acids are cholic acid and chenodeoxycholic acid (the “primary bile acids”). Before exiting the hepatocytes and forming bile, the bile acids are conjugated to either glycine (to produce glycocholic acid or glycochenodeoxycholic acid) or taurine (to produce taurocholic acid or taurochenodeoxycholic acid). The conjugated bile acids are called bile salts and their amphipathic nature makes them more efficient detergents than bile acids. Bile salts, not bile acids, are found in bile.

Bile salts are excreted by the hepatocytes into the canaliculi to form bile. The canaliculi drain into the right and left hepatic ducts and the bile flows to the gallbladder. Bile is released from the gallbladder and travels to the duodenum, where it contributes to the metabolism and degradation of fat. The bile salts are reabsorbed in the terminal ileum and transported back to the liver via the portal vein. Bile salts often undergo multiple enterohepatic circulations before being excreted via feces. A small percentage of bile salts may be reabsorbed in the proximal intestine by either passive or carrier-mediated transport processes. Most bile salts are reclaimed in the distal ileum by a sodium-dependent apically located bile acid transporter referred to as apical sodium-dependent bile acid transporter (ASBT). At the basolateral surface of the enterocyte, a truncated version of ASBT is involved in vectoral transfer of bile acids/salts into the portal circulation. Completion of the enterohepatic circulation occurs at the basolateral surface of the hepatocyte by a transport process that is primarily mediated by a sodium-dependent bile acid transporter. Intestinal bile acid transport plays a key role in the enterohepatic circulation of bile salts. Molecular analysis of this process has recently led to important advances in understanding of the biology, physiology, and pathophysiology of intestinal bile acid transport.

Within the intestinal lumen, bile acid concentrations vary, with the bulk of the reuptake occurring in the distal intestine. Described herein are certain compositions and methods that control bile acid concentrations in the intestinal lumen, thereby controlling the hepatocellular damage caused by bile acid accumulation in the liver.

A “rare disease” is a disease or condition that affects fewer than 1 in 200,000 people in the United States, or fewer than 65 in 100,000 people worldwide. A list of rare diseases and conditions can be accessed at, for example, www.rarediseases.org, which is incorporated by reference herein in its entirety.

In one aspect, the present disclosure provides methods of treating, reducing, or ameliorating cholestatic pruritus in a subject having a cholestatic liver disease.

In one embodiment, the cholestatic liver disease is a rare cholestatic liver disease.

In one embodiment, the rare cholestatic liver disease is selected from the group consisting of biliary atresia (BA), Caroli disease, Caroli syndrome, ciliopathies (Joubert syndrome, Meckel-Gruber syndrome, NPHP3 mutation), alpha-1-antitrypsin deficiency, chronic idiopathic hepatitis, chronic viral hepatitis, secondary sclerosing cholangitis (related to COVID-19 or injury), IgG4-related sclerosing cholangitis, ARC, Langerhans cell histiocytosis, sodium taurocholate co-transporting polypeptide deficiency, transaldolase deficiency, X-linked myotubular myopathy, hepatic sarcoidosis, idiopathic amyloidosis, ischemic cholangiopathy, rare metabolic disorders, nonalcoholic fatty liver disease, post-liver transplant cholestasis (including patients with ALGS, PBC, PFIC, or PSC who have had liver transplant), and undiagnosed cholestatic pruritus.

In one embodiment, the rare cholestatic liver disease is selected from the group consisting of Caroli disease, Caroli syndrome, ciliopathies (Joubert syndrome, Meckel-Gruber syndrome, NPHP3 mutation), alpha-1-antitrypsin deficiency, chronic idiopathic hepatitis, secondary sclerosing cholangitis (related to COVID-19 or injury), IgG4-related sclerosing cholangitis, ARC, Langerhans cell histiocytosis, sodium taurocholate co-transporting polypeptide deficiency, transaldolase deficiency, X-linked myotubular myopathy, hepatic sarcoidosis, idiopathic amyloidosis, ischemic cholangiopathy, rare metabolic disorders, post-liver transplant cholestasis (including patients with ALGS, PBC, PFIC, or PSC who have had liver transplant), and undiagnosed cholestatic pruritus.

In one embodiment, the rare cholestatic liver disease is not ALGS, PFIC, PBC, or PCS.

In one embodiment, the subject has a rare metabolic disorder selected from the group consisting of an organic acidemia, an amino acid disorder, a disorder of glucose metabolism, a congenital disorder of glycosylation (CDG), and a mitochondrial disorder. In one embodiment, the organic acidemias comprise, but are not limited to, methylmalonic acidemia and propionic acidemia. In one embodiment, the amino acid disorders comprise, but are not limited to, maple syrup urine disease and tyrosinemia. In one embodiment, the disorders of glucose metabolism comprise, but are not limited to, glycogen storage disease, galactosemia, and transaldolase deficiency. In one embodiment, the congenital disorders of glycosylation (CDGs) comprise, but are not limited to, disorders of protein N-glycosylation, disorders of protein O-glycosylation, disorders of glycosphingolipid and GPI-anchor glycosylation, and defects of multiple glycosylation. In one embodiment, the mitochondrial disorders include, but are not limited to, mitochondrial encephalomyopathy with lactic acidosis and stroke-like episodes (MELAS), mitochondrial neurogastrointestinal encephalomyopathy (MNGIE), and Pearson syndrome.

### Caroli Disease

Caroli disease, also known as Caroli's disease and type V choledochal cyst, is a rare genetic condition that causes the bile ducts in the liver to be wider than usual. Widening (dilation) of the bile ducts in the liver (intrahepatic bile ducts) can cause bile duct stones to form, which can lead to yellowing of the skin (jaundice) and flu-like symptoms. If the bile ducts become too wide, bile will begin to collect and can make the bile ducts inside of the liver swollen (cholangitis). Cholangitis can cause pain in the stomach, fever, tiredness, and nausea and vomiting. Caroli disease, when symptomatic, usually manifests with recurrent episodes of upper right quadrant abdominal pain, which may be accompanied by pruritus and jaundice. People with Caroli disease can have many episodes of these symptoms over their lifetime.

A different type of Caroli disease is called Caroli syndrome. It is common to group these two conditions together because they share features. Classic Caroli disease involves the malformation of the biliary tract, whereas Caroli syndrome refers to the presence of associated congenital hepatic fibrosis. People with Caroli syndrome have congenital hepatic fibrosis (liver scarring) which can cause high blood pressure in the veins in the liver (portal hypertension) as well as problems with the bile ducts in the liver. Sometimes, people with Caroli syndrome can develop polycystic kidney disease. Symptoms of Caroli syndrome are similar to Caroli disease, but can also include blood in the stools, frequent illnesses, and pain in the abdomen. Symptoms of these two conditions usually begin by the age of 30, but can happen at any age.

There is no current treatment for Caroli disease and Caroli syndrome. Medication such as antibiotics can be used to prevent irritation. Surgery is typically put off until symptoms start, since it is an invasive procedure. Surgery can be used to remove part of the liver where the bile ducts are too wide (hemi-hepatectomy) and if the patient is having too many episodes of cholangitis.

### Ciliopathies

Ciliopathies are complex disorders caused by genetic mutations which result in defective or dysfunctional cilia in many organs of the human body.

### Alström Syndrome

Alström Syndrome is a very rare recessively inherited condition which affects the metabolism of many major organs, particularly the heart, lungs, kidneys and liver.

Because the condition unfolds gradually from birth and the different manifestations vary from individual to individual, correct diagnosis is often delayed leading to suboptimal treatment and a failure to anticipate future developments. There are presently just over 1000 cases known worldwide although the history of poor diagnosis referred to above almost certainly hides many more.

### Bardet-Biedl Syndrome

Bardet-Biedl Syndrome (BBS) is a rare, recessively inherited complex disorder that involves many body systems. Mutations in more than 20 different genes encoding proteins involved in cilia formation, maintenance and signaling can cause BBS. Genetic screening is a major part of the diagnostic pathway. There is at present no cure for Bardet-Biedl syndrome. The incidence is estimated at 1 in 100,000 births.

### Joubert Syndrome (JBTS)

Joubert Syndrome is a rare developmental disorder affecting mainly the brain but this might be accompanied by renal and/or retinal symptoms. Mutations in several genes associated with cilia can cause Joubert Syndrome which are inherited often in a autosomal-recessive manner but x-chromosomal-recessive inheritance also occurs.

### Meckel-Gruber Syndrome

Meckel-Gruber syndrome is a rare autosomal recessive lethal malformation characterized by typical manifestations of occipital encephalocele, bilateral polycystic kidneys and post axial polydactyly. The worldwide incidence varies from 1 in 13,250 to 1 in 140,000 live births. Meckel-Gruber syndrome (MGS) is a triad of occipital encephalocele, large polycystic kidneys, and postaxial polydactyly. It is a rare, lethal autosomal recessive condition mapped to 6 different loci in different chromosomes. Meckel-Gruber syndrome is a condition characterized by ciliopathies caused by dysfunction of cilia and flagella. The high mortality and morbidity is due to non functional kidneys, liver and pulmonary hypoplasia.

### Nephronophthisis

Nephronophthisis (NPHP) is the most common genetic cause of chronic kidney disease within the first three decades of life. Presentation may occur during infancy but more typically in late childhood with progressive renal failure manifesting during early puberty.

Ultrasonographic features demonstrate normal size kidneys with loss of cortico-medullary differentiation and increased echogenicity. Histologically, NPHP kidneys are characterized by the presence of cortico-medullary cysts, tubular basement membrane disruption and tubulointerstitial nephropathy.

Inherited in an autosomal recessive mode, NPHP is genetically heterogeneous with at least 17 genes currently implicated which account for only about 30% of cases. NPHP is a ciliopathy disorder because similarly to the proteins involved in polycystic kidney disease, the nephrocystins (NPHP associated proteins) have all been localized to primary cilia, basal bodies and centrosomes. Many different ciliopathy patients show symptoms of NPHP.

### Primary Ciliary Dyskinesia

Primary Ciliary Dyskinesia (PDC) is the only known disorder of the motile cilia. However, dysfunction of motile cilia is implicated in several primary cilia ciliopathies—but these links are not yet well established. PCD is an autosomal recessive disorder which presents with upper and lower respiratory tract infection, and affects the lungs, sinuses and ears. Other features of PCD reflect dysfunction of cilia motility outside the airways, including subfertility, hydrocephalus and body laterality (left-right axis) defects. Some rare cases have retinal and neurological problems. The incidence is 1 in 7500. However, the incidence can be as high as 1 in 2,500 in the Asian population and other areas where consanguineous marriages are prevalent.

Recent studies have begun to locate PCD genes scattered throughout the genome, making it a heterogeneous condition. More than 50 different genes have been found to cause PCD, either with recessive inheritance and more rare X-linked-recessive inheritance. PCD affects both sexes and all populations. It is managed by physiotherapy and targeted antibiotics.

### Alpha-1 Antitrypsin Deficiency

Alpha-1 antitrypsin deficiency is an inherited disorder that may cause lung disease and liver disease. The signs and symptoms of the condition and the age at which they appear vary among individuals.

People with alpha-1 antitrypsin deficiency usually develop the first signs and symptoms of lung disease between ages 25 and 50. The earliest symptoms are shortness of breath following mild activity, reduced ability to exercise, and wheezing. Other signs and symptoms can include unintentional weight loss, recurring respiratory infections, and fatigue. Affected individuals often develop emphysema, which is a lung disease caused by damage to the small air sacs in the lungs (alveoli). Characteristic features of emphysema include difficulty breathing, a hacking cough, and a barrel-shaped chest. Smoking or exposure to tobacco smoke accelerates the appearance of emphysema symptoms and damage to the lungs.

About 10 percent of infants with alpha-1 antitrypsin deficiency develop liver disease, which often causes yellowing of the skin and whites of the eyes (jaundice). Approximately 15 percent of adults with alpha-1 antitrypsin deficiency develop liver damage (cirrhosis) due to the formation of scar tissue in the liver. Signs of cirrhosis include a swollen abdomen and jaundice. Individuals with alpha-1 antitrypsin deficiency are also at risk of developing a type of liver cancer called hepatocellular carcinoma. Other symptoms may include swelling of the legs or belly (ascites), blood in the stool, and pruritus.

### Chronic Viral Hepatitis

Viral hepatitis is an infection that causes liver inflammation and damage. Inflammation is swelling that occurs when tissues of the body become injured or infected. Inflammation can damage organs. Researchers have discovered several different viruses that cause hepatitis, including hepatitis A, B, C, D, and E.

Hepatitis A and hepatitis E typically spread through contact with food or water that has been contaminated by an infected person's stool. People may also get hepatitis E by eating undercooked pork, deer, or shellfish. The hepatitis A and E viruses typically cause only acute, or short-term, infections.

Hepatitis B, hepatitis C, and hepatitis D spread through contact with an infected person's blood. Hepatitis B and D may also spread through contact with other body fluids. This contact can occur in many ways, including sharing drug needles or having unprotected sex.

The hepatitis B, C, and D viruses can cause acute and chronic, or long-lasting, infections. Chronic hepatitis occurs when your body isn't able to fight off the hepatitis virus and the virus does not go away. Chronic hepatitis can lead to complications such as cirrhosis, liver failure, and liver cancer. Early diagnosis and treatment of chronic hepatitis can reduce chances of developing these complications.

When doctors can't find the cause of a person's hepatitis, they may call this condition non-A-E hepatitis or hepatitis X. Experts think that unknown viruses other than hepatitis A, B, C, D, and E may cause some cases of hepatitis. Researchers are working to identify these viruses. Although non-A-E hepatitis is most often acute, it can become chronic.

### Chronic Idiopathic Hepatitis

Chronic idiopathic hepatitis is defined as chronic necroinflammatory self-perpetuating liver disease associated with a nonsuppurative inflammatory infiltrate. To qualify as an idiopathic syndrome, an underlying cause should have been rigorously pursued yet not discovered.

### Biliary Atresia

Biliary atresia is a life-threatening condition in infants in which the bile ducts inside or outside the liver do not have normal openings. With biliary atresia, bile becomes trapped, builds up, and damages the liver. The damage leads to scarring, loss of liver tissue, and cirrhosis. Without treatment, the liver eventually fails and the infant needs a liver transplant to stay alive. The two types of biliary atresia are fetal and perinatal. Fetal biliary atresia appears while the baby is in the womb. Perinatal biliary atresia is much more common and does not become evident until 2 to 4 weeks after birth.

### Post-Kasai Biliary Atresia

Biliary atresia is treated with surgery called the Kasai procedure or a liver transplant. The Kasai procedure is usually the first treatment for biliary atresia. During a Kasai procedure, the pediatric surgeon removes the infant's damaged bile ducts and brings up a loop of intestine to replace them. While the Kasai procedure can restore bile flow and correct many problems caused by biliary atresia, the surgery doesn't cure biliary atresia. If the Kasai procedure is not successful, infants usually need a liver transplant within 1 to 2 years. Even after a successful surgery, most infants with biliary atresia slowly develop cirrhosis over the years and require a liver transplant by adulthood. Possible complications after the Kasai procedure include ascites, bacterial cholangitis, portal hypertension, and pruritis.

### Post Liver Transplantation Biliary Atresia

If the atresia is complete, liver transplantation is the only option. Although liver transplantation is generally successful at treating biliary atresia, liver transplantation may have complications such as organ rejection. Also, a donor liver may not become available. Further, in some patients, liver transplantation may not be successful at curing biliary atresia.

### Secondary Sclerosing Cholangitis

Secondary sclerosing cholangitis (SSC) may arise from a variety of causes presenting with biliary strictures, both benign and malignant. The most common benign causes are iatrogenic and secondary to biliary injury after cholecystectomy or liver transplant.

SSC is a disease with morphologic, radiologic, and clinical features that is similar to PSC but in which the underlying cause of ductal inflammation and sclerosis is known. Several pathologic processes may lead to secondary sclerosing cholangitis; recognition and exclusion of these entities is pivotal in differentiating secondary from primary sclerosing cholangitis. Posttraumatic sclerosing cholangitis (SSC following an injury) is a rare but rapidly progressive disease, probably caused by ischemia of the intrahepatic bile ducts via the peribiliary capillary plexus due to arterial hypotension. Gastroenterologists should be aware of this disease in patients with persistent cholestasis after severe trauma.

The coronavirus disease 2019 (COVID-19) pandemic has had a profound impact on global health, primarily characterized by severe respiratory illness. However, emerging evidence suggests that COVID-19 can also lead to secondary sclerosing cholangitis (SSC), also referred to as post-COVID-19 cholangiopathy. It is characterized by inflammation and damage to the bile ducts. Following prolonged stays in the intensive care unit, these patients develop marked sustained cholestasis and jaundice despite clinical improvement. Cholangiography shows beaded appearance of intra-hepatic bile ducts and bile casts. None of the patients reach normalization of liver enzymes and some progressed to liver cirrhosis (follow-up time of 11 to 16 months). COVID-19-associated SSC has a dismal prognosis with rapid progression to advanced chronic liver disease. Post-COVID-19 cholangiopathy is a serious condition that is expected to become increasingly concerning in the coming years, particularly considering long COVID syndromes. Although liver transplantation has been proposed as a potential treatment option, more research is necessary to establish its efficacy and explore other potential treatments.

### IgG4-Related Sclerosing Cholangitis

IgG4-related sclerosing cholangitis (IgG4-SC) is a biliary stricturing disease caused by an IgG4-predominant subepithelial lymphoplasmacytic infiltration of intrahepatic and extrahepatic bile ducts. IgG4-SC is clinically distinct from primary sclerosing cholangitis (PSC). IgG4-SC can manifest with cholangitis and pancreatitis. Diagnosis requires an abnormal cholangiogram, elevated serum IgG4 levels, and histologic analysis. Although treatment with corticosteroids may lead to remission, there is currently no treatment for the underlying condition.

### Arthrogryposis Renal Dysfunction Cholestasis Syndrome (ARC)

Arthrogryposis renal dysfunction cholestasis syndrome (ARC) is a very rare genetic condition. The condition is present at birth, but symptoms may not be noticed until later in a child's life. There are two types of ARC, each caused by variants in a specific gene, but the signs and symptoms are similar for both types. ARC type 1 is caused by variants in the VIPAS39 gene. ARC type 2 is caused by variants in the VPS33B gene.

While it has been thought that babies affected with ARC do not survive beyond the first year of life, usually dying from sepsis, dehydration or acidosis, we now know that there are “attenuated” or mild cases. There are children now enrolled in elementary school who are known to have ARC type 1. The classical presentation of ARC includes congenital joint contractures, renal tubular dysfunction, and cholestasis. Additional features include ichthyosis, central nervous system malformation, platelet anomalies, and severe failure to thrive. Diagnosis of ARC syndrome relies on clinical features, organ biopsy, and mutational analysis. However, no specific treatment currently exists for this syndrome.

### Langerhans Cell Histiocytosis (LCH)

Langerhans cell histiocytosis (LCH) is an idiopathic condition characterized by proliferation of abnormal Langerhans (antigen-presenting) cells. The disease has characteristics of both an abnormal reactive process and a neoplastic process. It may present initially as a rash. It can be disseminated and involve the bone marrow, lungs, liver, spleen, lymph nodes, gastrointestinal tract, and pituitary gland. Prognosis varies depending on presentation and organ involvement. LCH can occur at any age but is most common from birth to age 15 years. There is a wide variation in presentation, treatment, and prognosis. Referral and long-term follow up with an oncologist are important in the management of LCH.

### Sodium Taurocholate Co-Transporting Polypeptide (NTCP) Deficiency

Sodium-taurocholate co-transporting polypeptide (NTCP) deficiency is a newly reported hereditary bile acid metabolic disease. NTCP is encoded by the solute carrier family 10 member 1 (SLC10A1) gene with a total length of 23 kbp, localized in chromosome 14q24.2, which contains 5 exons. The protein product NTCP is composed of 349 amino acid residues, with a molecular weight of 38 kDa. NTCP is localized at the basolateral membrane of hepatocytes. It is a sodium-dependent transporter that is involved in the transport of bile acids from the blood to the hepatocytes in order to maintain the uninterrupted enterohepatic circulation of bile salts, and its deficiency leads to elevated levels of circulating bile acids (hypercholanemia). The clinical and biochemical influences of hypercholanemia are not fully understood. As of November 2020, a total of 60 cases of pediatric NTCP deficiency had been reported in 11 studies. The common feature is hypercholanemia without obvious symptoms and the mode of inheritance is autosomal recessive. However, the clinical features need to be further defined, especially in pediatric patients, in order to promote early recognition and avoid unnecessary intervention and anxiety. Confirmation of the diagnosis requires genetic analysis for homozygous or compound heterozygous mutations of SLC10A1. Further study is needed to interpret the mechanisms of hyperbilirubinemia, elevated AST, and the absorption of fat soluble vitamins.

### Transaldolase Deficiency

Transaldolase deficiency is an inborn error of the pentose phosphate pathway that presents in the neonatal or antenatal period with hydrops fetalis, hepatosplenomegaly, hepatic dysfunction, thrombocytopenia, anemia, and renal and cardiac abnormalities. Less than ten cases have been reported in the literature so far. The disorder is caused by mutations in the transaldolase gene (TALDO1, 11p15.5-p15.4). Dysmorphic features (downward-slanting palpebral fissures, low-set ears, and cutis laxa) have also been described. The severity of the symptoms and outcome vary widely.

### X-Linked Myotubular Myopathy

X-linked myotubular myopathy (XLMTM or MTM) is caused by a genetic mutation on the X chromosome. It occurs almost exclusively in males, affecting about 1 in 50,000 newborn boys worldwide. XLMTM is a rare genetic neuromuscular disorder that is characterized by muscle weakness that is most typically severe but can range from mild to profound. Symptoms are often present at birth, though may develop later in infancy or early childhood. Rarely, symptoms may not present until adolescence or adulthood. Common symptoms include mild to profound muscle weakness, diminished muscle tone (hypotonia or “floppiness”), feeding difficulties, and potentially severe breathing complications (respiratory distress). Feeding difficulties and respiratory distress develop because of weakness of the muscles that are involved in swallowing and breathing. The overall severity of the disorder can range from mildly affected individuals to individuals who develop severe, life-threatening complications during infancy and early childhood. Most affected individuals have a severe form of the disorder and respiratory failure is an almost uniform occurrence. XLMTM is caused by mutations to the myotubularin (MTM1) gene. The disorder is inherited as an X-linked condition. The disorder predominantly affects males, but female carriers, while typically asymptomatic, can develop a range of symptoms. In rare specific cases, females can develop a severe form similar to that seen in males.

### Hepatic Sarcoidosis

Sarcoidosis is a multisystem disease characterized by the presence of non-caseating granulomas in affected organs. Pulmonary involvement is the most common site of disease activity. However, hepatic involvement is also common in sarcoidosis, occurring in up to 70% of patients. Most patients with liver involvement are asymptomatic. Therefore, the majority of cases are discovered incidentally, frequently by the finding of elevated liver enzymes. Pain in the right upper quadrant of the abdomen, fatigue, pruritus, and jaundice may be associated with liver involvement. Portal hypertension and cirrhosis are complications linked to long-standing hepatic sarcoidosis. Liver biopsy is usually required to confirm the diagnosis. It is important to differentiate hepatic sarcoidosis from other autoimmune and granulomatous liver diseases. For symptomatic patients, the first line treatment includes corticosteroids or ursodeoxycholic acid. Various immunosuppressant agents can be used as second line agents. Rarely, severe cases require liver transplantation.

### Idiopathic Amyloidosis

Amyloidosis is a rare disease characterized by a buildup of abnormal amyloid deposits in the body. Amyloid deposits can build up in the heart, brain, kidneys, spleen and other parts of the body. A person may have amyloidosis in one organ or several. Amyloidosis may be secondary to a different health condition or can develop as a primary condition. Sometimes it is due to a mutation in a gene, but other times the cause of amyloidosis remains unknown (idiopathic).

Light-chain (AL) amyloidosis can affect the kidneys, spleen, heart, and other organs. People with conditions such as multiple myeloma or a bone marrow illness called Wadenström's macroglobulinemia are more likely to have AL amyloidosis. AL starts in plasma cells within the bone marrow. Plasma cells create antibodies with both heavy chain and light chain proteins. If the plasma cells undergo abnormal changes, they produce excess light chain proteins that can end up in the bloodstream. These damaged protein bits can accumulate in the body's tissues and damage vital organs such as the heart.

AA amyloidosis is caused by fragments of amyloid A protein, and affects the kidneys in about 80 percent of cases. It can complicate chronic diseases characterized by inflammation, such as rheumatoid arthritis (RA) or inflammatory bowel disease (IBS).

Transthyretin amyloidosis (ATTR) can be inherited from a family member (familial amyloidosis). People of African descent may be more likely to carry the gene that causes this kind of amyloidosis. Transthyretin is a protein that is also known as prealbumin. It is made in the liver. Excessive normal (wild-type ATTR) or mutant transthyretin can cause amyloid deposits.

There are other forms of amyloidosis, including APOA1, gelsolin, fibrinogen, and lysozyme.

### Ischemic Cholangiopathy

Ischemic cholangiopathy is defined as a focal or extensive damage to the bile ducts due to impaired blood supply. Bile ducts (such as the hepatic ducts and the common bile duct), unlike the liver, are supplied with blood from only one major blood vessel, the hepatic artery. Thus, disruption of blood flow through the hepatic artery can prevent the bile ducts from obtaining enough oxygen. Consequently, the cells lining the ducts are damaged or die—a disorder called ischemic cholangiopathy. Blood flow can be disrupted when: 1) a liver transplant is rejected; 2) blood vessels are injured during liver transplantation surgery or removal of the gallbladder by laparoscopy; 3) blood vessels are injured by radiation therapy; 4) patient has a blood clotting disorder; 5) a procedure that is done to block blood flow to a tumor in the liver (chemoembolization) also blocks blood flow to healthy tissue. Ischemic cholangiopathy most commonly occurs in people who have had a liver transplant.

Pruritus is common, often beginning in the hands and feet but usually affecting the whole body. Itching is especially worse at night.

### Nonalcoholic Fatty Liver Disease

Nonalcoholic fatty liver disease (NAFLD) is a condition in which fat builds up in your liver. NAFLD is also known as Nonalcoholic Steatotic Liver Disease (NASLD), Metabolic Dysfunction-Associated Fatty Liver Disease (MAFLD), and Metabolic Dysfunction-Associated Steatotic Liver Disease (MASLD), which terms are used interchangeably herein. Nonalcoholic fatty liver (NAFL) and nonalcoholic steatohepatitis (NASH) are types of NAFLD. NASH is characterized by inflammation and liver damage, along with fat presence in the liver. Certain health conditions and diseases, including obesity, metabolic syndrome, and type 2 diabetes, increase the chances of developing NAFLD. When symptoms are present, they usually include pain in the upper right side of the abdomen, fatigue/weakness, weight loss, fluid and swelling in the stomach (ascites) and legs (edema), jaundice, or yellowing of the skin and eyes, and pruritus.

### Post-Liver Transplant Cholestasis

In patients after liver transplantation, interference in the uptake, transfer, and secretion of bile caused by hepatocyte and/or cholangiocyte cell injury can result in cholestasis. Although the majority of such events remain subclinical, severe cholestasis may be associated with irreversible liver damage requiring retransplantation. Post-transplantation cholestasis may be extrahepatic, involving a mechanical obstruction of the main bile ducts, or intrahepatic, involving impairment of bile secretion because of a defect in the hepatocytes or microscopic bile ducts within the liver. Causes of intrahepatic cholestasis may be categorized by time of occurrence, namely, within 6 months of liver transplantation (early) and thereafter (late), although there may be an overlap in their causes. The causes of intrahepatic cholestasis include ischemia/reperfusion injury, bacterial infection, acute cellular rejection, cytomegalovirus infection, small-for-size graft, drugs for hepatotoxicity, intrahepatic biliary strictures, chronic rejection, hepatic artery thrombosis, ABO blood group incompatibility, and recurrent disease. In some cases, patients with ALGS, PBC, PFIC, or PSC who have had liver transplant may develop post-liver transplant cholestasis.

### Organic Acidemias

Organic acidemias, also known as organic acidurias, are a class of heterogeneous inherited metabolic disorders characterized by accumulation of abnormal (and usually toxic) organic acid metabolites and increased excretion of organic acids in urine. They result primarily from deficiencies of specific enzymes in the breakdown pathways of amino acids. Most organic acidemias become clinically apparent during the newborn period or early infancy, although there are milder forms that may not present until adolescence or adulthood or not come to medical attention at all. After an initial period of well-being, affected children may develop a life-threatening episode of metabolic acidosis characterized by an increased anion gap. This presenting episode may often be mistaken for sepsis and, if unrecognized, is associated with significant mortality. Metabolic decompensation can occur during episodes of increased catabolism, such as intercurrent illness, trauma, surgery, or prolonged episodes of fasting. In one embodiment, the organic acidemias comprise, but are not limited to, methylmalonic acidemia and propionic acidemia.

Methylmalonic acidemia (MMA) is a rare, genetic disorder of the liver. People with this condition are unable to produce an enzyme that is needed to break down and use certain proteins and fats found in food. The effects of methylmalonic acidemia, which usually appear in early infancy, vary from mild to life-threatening. Affected infants can experience vomiting, dehydration, weak muscle tone (hypotonia), developmental delays, excessive tiredness (lethargy), an enlarged liver (hepatomegaly), and failure to gain weight and grow at the expected rate (failure to thrive). Long-term complications can include feeding problems, intellectual disabilities, movement problems, chronic kidney disease, and inflammation of the pancreas (pancreatitis). People with methylmalonic acidemia can have frequent episodes of excess acid in the blood (metabolic acidosis) that cause serious health complications. Without treatment, this disorder can lead to coma and death in some cases.

Propionic acidemia is a rare metabolic disorder affecting from 1/20,000 to 1/250,000 individuals in various regions of the world. It is characterized by deficiency of propionyl-CoA carboxylase, an enzyme involved in the breakdown (catabolism) of the chemical “building blocks” (amino acids) of proteins. Symptoms most commonly become apparent during the first weeks of life and may include abnormally diminished muscle tone (hypotonia), poor feeding, vomiting, listlessness (lethargy), dehydration and seizures. Without appropriate treatment, coma and death may result. Rarely, the condition may become apparent later in life and may be associated with less severe symptoms and findings. Propionic acidemia is inherited in an autosomal recessive pattern. Individuals with this condition have to follow a specific diet including a low protein intake and specific food formulas (medical foods). Liver transplant is a surgical option that can help decrease the frequency of acute metabolic episodes (decompensation).

### Amino Acid Disorders

Amino acid disorders are a group of metabolic disorders that affect the body's ability to use proteins from food for growth, energy, and repair. Amino acids are necessary building blocks of proteins but can be harmful when they build up in the body. With amino acid disorders, certain amino acids build up since key enzymes are not produced by the body or do not work properly. Amino acid disorders include, but are not limited to, argininemia, argininosuccinic aciduria, benign hyperphenylalaninemia, biopterin defect in cofactor biosynthesis, biopterin defect in cofactor regeneration, carbamoylphosphate synthetase deficiency, citrullinemia type 1, citrullinemia type 2, phenylketonuria, gyrate atrophy of the choroid and retina, homocystinuria, hypermethioninemia, hyperornithinemia-hyperammonemia-homocitrullinuria syndrome, hyperprolinemia type 1, hyperprolinemia type 2, maple syrup urine disease, ornithine transcarbamylase deficiency, remethylation defects (MTHFR, MTR, MTRR, Cbl D v1, Cbl G deficiencies), and tyrosinemia (including tyrosinemia type 1, tyrosinemia type 2, tyrosinemia type 3, and transient tyrosinemia). In one embodiment, the amino acid disorders comprise, but are not limited to, maple syrup urine disease and tyrosinemia.

Maple syrup urine disease (MSUD) is named for the sweet maple syrup smell of the urine in untreated babies. It is one type of amino acid disorder. People with this type of disorder have problems breaking down amino acids from the protein in the food. Classic MSUD is caused by the absence of a group of enzymes called “branched-chain ketoacid dehydrogenase” (BCKAD). This enzyme group breaks down three different amino acids: leucine, isoleucine, and valine. When they cannot be broken down these amino acids build up in the blood. These amino acids are “branched-chain amino acids” (BCAA) and are found in all foods that have protein. Without treatment, severe, life-threatening symptoms can develop, including seizures (fits) or falling into a coma. Some children with untreated MSUD are also at risk of brain damage and developmental delay.

Tyrosinemia is a genetic disorder characterized by problems breaking down the amino acid tyrosine, which is a building block of most proteins. If the condition is untreated, tyrosine and its byproducts build up in tissues and organs, which can lead to serious health problems. There are three types of tyrosinemia, distinguished by their symptoms and genetic cause.

Tyrosinemia type I is the most severe form of this disorder and usually begins in the first few months of life. Affected infants do not gain weight and grow at the expected rate (failure to thrive) because eating high-protein foods leads to diarrhea and vomiting. Affected infants may also have yellowing of the skin and whites of the eyes (jaundice), a cabbage-like odor, and an increased tendency to bleed (particularly nosebleeds). In addition, tyrosinemia type I can lead to liver and kidney failure, softening and weakening of the bones (rickets), and an increased risk of liver cancer (hepatocellular carcinoma). Some affected children have repeated neurologic crises that consist of changes in their mental state, reduced sensation in the arms and legs (peripheral neuropathy), abdominal pain, and serious breathing problems (respiratory failure). These crises can last from 1 to 7 days. Without treatment, children with tyrosinemia type I often do not survive past the age of 10. With early diagnosis and treatment, though, affected individuals can live into adulthood.

Tyrosinemia type II often begins in early childhood and affects the eyes, skin, and mental development. Signs and symptoms include eye pain and redness, excessive tearing, abnormal sensitivity to light (photophobia), and thick, painful skin on the palms of the hands and soles of the feet (palmoplantar hyperkeratosis). About half of individuals with tyrosinemia type II have some degree of intellectual disability.

Tyrosinemia type III is the rarest of the three types. The characteristic features of this type include intellectual disabilities, seizures, and periodic loss of balance and coordination (intermittent ataxia). Liver problems do not occur in types II and III.

About 1 in 10 of all newborns have temporarily elevated levels of tyrosine (transient tyrosinemia). These cases are not genetic. The most likely causes are vitamin C deficiency or an immature liver due to premature birth.

### Disorders of Glucose Metabolism

Carbohydrate metabolism disorders or glucose metabolism disorders are a group of metabolic disorders. Normally enzymes break carbohydrates down into glucose (a type of sugar). Patients with one of these disorders may not have enough enzymes to break down the carbohydrates, or the enzymes may not work properly. This causes a harmful amount of sugar to build up in the body. That can lead to health problems, some of which can be serious. Some of the disorders are fatal.

These disorders are inherited. Newborn babies get screened for many of them, using blood tests. If there is a family history of one of these disorders, parents can get genetic testing to see whether they carry the gene. Other genetic tests can tell whether the fetus has the disorder or carries the gene for the disorder.

Treatments may include special diets, supplements, and medicines. Some babies may also need additional treatments, if there are complications. For some disorders, there is no cure, but treatments may help with symptoms. In one embodiment, the disorders of glucose metabolism comprise, but are not limited to, glycogen storage disease, galactosemia, and transaldolase deficiency.

Glycogen storage disease (GSD) is a rare condition that changes the way the body uses and stores glycogen, a form of sugar or glucose. Glycogen is a main source of energy for the body. Glycogen is stored in the liver. When the body needs more energy, certain proteins called enzymes break down glycogen into glucose. They send the glucose out into the body. When someone has GSD, they are missing one of the enzymes that breaks down glycogen. When an enzyme is missing, glycogen can build up in the liver. Or glycogen may not form properly. This can cause problems in the liver or muscles, or other parts of the body. GSD is passed down from parents to children (is hereditary). It is most often seen in babies or young children. But some forms of GSD may appear in adults.

Types of GSD are grouped by the enzyme that is missing in each one. Each GSD has its own symptoms and needs different treatment. There are several types of GSD, but the most common types are types I, III, and IV. These types are also known by other names:

Type I or von Gierke disease is the most common form of GSD. People with type I GSD don't have the enzyme needed to turn glycogen into glucose in the liver. Glycogen builds up in the liver. Symptoms often appear in babies around 3 to 4 months old. They may include low blood sugar (hypoglycemia) and a swollen belly because of an enlarged liver.

Type III is known as Cori disease, or Forbes disease. People with type III don't have enough of an enzyme called the debranching enzyme, which helps break down glycogen. The glycogen can't fully break down. It collects in the liver and in muscle tissues. Symptoms include a swollen belly, delayed growth, and weak muscles.

Type IV is also known as Andersen disease. People with type IV form abnormal glycogen. Experts think the abnormal glycogen triggers the body's infection-fighting system (immune system). This creates scarring (cirrhosis) of the liver and other organs such as muscle and the heart.

Galactosemia is a rare, hereditary disorder of carbohydrate metabolism that affects the body's ability to convert galactose to glucose. Galactose is a sugar contained in milk, including human mother's milk as well as other dairy products. It is also produced by the human body, and this is called endogenous galactose. Glucose is a different type of sugar. The disorder is caused by a deficiency of an enzyme galactose-1-phosphate uridylyl transferase (GALT) which is vital to this process. Early diagnosis and treatment with a lactose-restricted (dairy-free) diet is absolutely essential to avoid profound intellectual disability, liver failure and death in the newborn period. Galactosemia is inherited as an autosomal recessive genetic condition. Classic galactosemia and clinical variant galactosemia can both result in life-threatening health problems unless lactose is removed from the diet shortly after birth. A biochemical variant form of galactosemia termed Duarte is not thought to cause clinical disease due to lactose consumption.

Transaldolase (TALDO) deficiency is an inborn error of the pentose phosphate pathway, which is a severe, early-onset multisystem disease. The body fluids of affected patients contain increased concentrations of polyol, heptulose, sedoheptulose, mannoheptulose and sedoheptulose-7P, mostly in the urine.

Patients present severe symptoms during the neonatal period, and in almost all cases, some signs were already noted prenatally. The leading symptoms in transaldolase-deficient patients are anaemia, bleeding problems with thrombocytopenia, hepatosplenomegaly, nodular progressive hepatic fibrosis and later on nephropathy. To date, 23 patients from 13 families have been described. The majority of patients have consanguineous parents.

### Congenital Disorders of Glycosylation (CDG)

Congenital disorders of glycosylation (CDGs) is an umbrella term for a rapidly expanding group of over 130 rare genetic, metabolic disorders due to defects in a complex chemical process known as glycosylation. Glycosylation is the process by which sugar ‘trees’ (glycans) are created, altered and attached to 1000's of proteins or fats (lipids). When these sugar molecules are attached to proteins, they form glycoproteins; when they are attached to lipids, they form glycolipids. Glycoproteins and glycolipids have numerous important functions in all tissues and organs. Glycosylation involves many different genes, encoding many different proteins such as enzymes. A deficiency or lack of one of these enzymes can lead to a variety of symptoms potentially affecting multiple organ systems. CDG can affect any part of the body and there is nearly always an important neurological component. CDG can be associated with a broad variety of symptoms and can vary in severity from mild to severe, disabling or life-threatening. CDG are usually apparent from infancy. Individual CDG are caused by changes (mutations) in a specific gene. Most CDG are inherited as autosomal recessive conditions, but some are X-linked or dominant. Others may arise spontaneously (de novo).

In one embodiment, the congenital disorders of glycosylation (CDGs) comprise, but are not limited to, disorders of protein N-glycosylation, disorders of protein O-glycosylation, disorders of glycosphingolipid and GPI-anchor glycosylation, and defects of multiple glycosylation. A classification system for CDGs exists that names each type by the official abbreviation of the abnormal gene followed by a dash and CDG. For example, congenital disorder of glycosylation type 1a is now known as PMM2-CDG because a mutation in the PMM2 gene causes this type of CDG. Major categories of CDG are based on the glycosylation pathway or molecule that is affected. For instance, disorders of protein glycosylation are broken down into two groups known as disorders of N-glycosylation and disorders of O-glycosylation. Other types of CDG include disorders of glycosphingolipid and GPI-anchor glycosylation, and disorders of multiple glycosylation and other pathways. These four categories of CDG are described below.

Most types of CDG are classified as disorders of N-glycosylation, which involve carbohydrates called N-linked oligosaccharides (glycans). Disorders of N-glycosylation are due to an enzyme deficiency or other malfunction somewhere along the N-glycosylation pathway. This category of CDG can be further divided into two subtypes: defects of oligosaccharide assembly and transfer (type 1) and defects in oligosaccharide trimming and processing that occur after they are bound to proteins (type 2). Disorders of protein N-glycosylation notably include PMM2-CDG, the most common type of CDG.

Disorders of O-glycosylation are due to an enzyme deficiency or other malfunction somewhere along the O-glycosylation pathway. Some of these disorders are better known than the N-linked forms and many have more traditional names. In some cases, they have also been classified as subtypes of other umbrella groups. For instance, some disorders of O-linked glycosylation are also classified as forms of muscular dystrophy. These disorders are collectively termed the dystroglycanopathies. The NORD database has individual reports on Walker-Warburg syndrome and Fukuyama muscular dystrophy, as well as general overviews on congenital muscular dystrophy and limb-girdle muscular dystrophy. Other disorders involve defects in the synthesis of large molecules called glycosaminoglycans (GAGs) which are the sugar components of proteoglycans.

As their name indicates, these disorders involve defects in the glycosylation of two types of lipid-containing molecules: glycosphingolipids (GSL) and glycosylphosphatidylinositol (GPI) anchors. These glycolipids have a wide range of functions in the body. Disorders associated with a defect in their production can therefore have a wide range of manifestations, as it is the case for disorders of protein glycosylation. There are over 20 types of GPI anchor disorders, but just a couple in GSL synthesis.

Some CDG occur due to defects that impact and alter multiple glycosylation pathways. For example, some individuals may have defects affecting both the N-linked and O-linked glycosylation pathways. These can also include defects in the organization, delivery, or trafficking of proteins within cells. These disorders usually have manifestations similar to other categories of congenital disorders of glycosylation.

### Mitochondrial Disorders

Mitochondrial disorders are caused by defects in mitochondria, which are energy factories found inside almost all the cells in the body. There are many types of mitochondrial disorders. They can affect one part of the body or many parts, including the brain, muscles, kidneys, heart, eyes, and ears. Symptoms of mitochondrial disorders vary because a person can have a unique mixture of healthy and defective mitochondria, with a unique distribution of each within the body. In most cases, mitochondrial disorders affect more than one type of cell, tissue, or organ. Because muscle and nerve cells have especially high energy needs, muscular and neurological problems are common features of mitochondrial disorders. Other common symptoms include impaired vision, hearing loss, abnormal heartbeat (cardiac arrhythmia), diabetes, and stunted growth.

Mitochondrial disorders that mostly cause muscular problems are called mitochondrial myopathies (“myo” means muscle and “pathos” means disease), while mitochondrial disorders that cause both muscular and neurological problems are called mitochondrial encephalomyopathies (encephalo refers to the brain). In one embodiment, the mitochondrial disorders include, but are not limited to, mitochondrial encephalomyopathy with lactic acidosis and stroke-like episodes (MELAS), mitochondrial neurogastrointestinal encephalomyopathy (MNGIE), and Pearson syndrome.

Mitochondrial encephalomyopathy often includes some symptoms of myopathy, plus one or more neurological symptoms. In addition to affecting the muscles around the eye, mitochondrial encephalomyopathy can affect the eye itself and parts of the brain involved in vision. For instance, vision loss, is a common symptom of mitochondrial encephalomyopathy. This can be caused by shrinkage of the optic nerve or a breakdown of the cells that line the back of the eye. Other common symptoms of mitochondrial encephalomyopathy include migraine headaches and seizures. There are many effective medications for treating and helping to prevent migraines and seizures, including anticonvulsants and other drugs developed to treat epilepsy.

Hearing loss is another common symptom of mitochondrial disorders. It is caused by damage to the inner ear or to the auditory nerve, which connects the inner ear to the brain. This kind of hearing loss is permanent, but it can be managed. Alternative forms of communication (like sign language), hearing aids, or cochlear implants can help. Mitochondrial disorders can cause ataxia, which refers to trouble with balance and coordination. People with ataxia are prone to falls and may need to use supportive aids such as railings, a walker, or a wheelchair. Physical and occupational therapy also may help. In some cases, mitochondrial disorders can lead to issues with breathing, heart health, kidney issues, diabetes, or digestive problems. People with mitochondrial disorders should get regular health check-ups to identify and monitor these potential problems.

Mitochondrial encephalomyopathy with lactic acidosis and stroke-like episodes (MELAS) syndrome is a rare maternally inherited mitochondrial disorder that predominantly affects the nervous system and muscles. MELAS typically appears in childhood after a period of normal early development. This condition manifests with recurrent episodes of encephalopathy, myopathy, headache, and focal neurological deficits in children or young adults, usually between the ages of 2 and 15. A distinctive feature of the syndrome is the occurrence of stroke-like episodes leading to hemiparesis, hemianopia, or cortical blindness.

A nucleotide substitution in transfer RNA (tRNA) is responsible for most cases of the disease. One specific substitution, the m.3243A>G (A-to-G substitution at nucleotide 3243), is responsible for 80% of cases, whereas another tRNA variation, the m.3271T>C (T-to-C substitution at nucleotide 3271), accounts for the remaining cases. MELAS is characterized by progressive deterioration of the nervous system that leads to neurological impairment and dementia in adolescence or early adulthood. Unfortunately, there is currently no known treatment that can slow or halt the progression of the disease.

Mitochondrial neurogastrointestinal encephalomyopathy (MNGIE) is a rare autosomal recessive disease caused by TYMP mutations and thymidine phosphorylase (TP) deficiency. Thymidine and deoxyuridine accumulate impairing the mitochondrial DNA maintenance and integrity. Clinically, patients show severe and progressive gastrointestinal and neurological manifestations. The onset typically occurs in the second decade of life and mean age at death is 37 years. Signs and symptoms of MNGIE are heterogeneous and confirmatory diagnostic tests are not routinely performed by most laboratories, accounting for common misdiagnosis. Factors predictive of progression and appropriate tests for monitoring are still undefined. Several treatment options showed promising results in restoring the biochemical imbalance of MNGIE. The lack of controlled studies with appropriate follow-up accounts for the limited evidence informing diagnostic and therapeutic choices.

Pearson syndrome is a severe disorder that usually begins in infancy. It causes problems with the development of blood-forming (hematopoietic) cells in the bone marrow that have the potential to develop into different types of blood cells. For this reason, Pearson syndrome is considered a bone marrow failure disorder. Function of the pancreas and other organs can also be affected. Most affected individuals have a shortage of red blood cells (anemia), which can cause pale skin (pallor), weakness, and fatigue. Some of these individuals also have low numbers of white blood cells (neutropenia) and platelets (thrombocytopenia). Neutropenia can lead to frequent infections; thrombocytopenia sometimes causes easy bruising and bleeding. When visualized under the microscope, bone marrow cells from affected individuals may appear abnormal. Often, early blood cells (hematopoietic precursors) have multiple fluid-filled pockets called vacuoles. In addition, red blood cells in the bone marrow can have an abnormal buildup of iron that appears as a ring of blue staining in the cell after treatment with certain dyes. These abnormal cells are called ring sideroblasts.

In people with Pearson syndrome, the pancreas does not work as well as usual. The pancreas produces and releases enzymes that aid in the digestion of fats and proteins. Reduced function of this organ can lead to high levels of fats in the liver (liver steatosis). The pancreas also releases insulin, which helps maintain correct levels of blood glucose, also called blood sugar. A small number of individuals with Pearson syndrome develop diabetes, a condition characterized by abnormally high blood glucose levels that can be caused by a shortage of insulin. In addition, affected individuals may have scarring (fibrosis) in the pancreas.

People with Pearson syndrome have a reduced ability to absorb nutrients from the diet (malabsorption), and most affected infants have an inability to grow and gain weight at the expected rate (failure to thrive). Another common occurrence in people with this condition is buildup in the body of a chemical called lactic acid (lactic acidosis), which can be life-threatening. In addition, liver and kidney problems can develop in people with this condition. Some people with Pearson syndrome have droopy eyelids (ptosis), vision problems, hearing loss, seizures, or movement disorders. About half of children with this severe disorder die in infancy or early childhood due to severe lactic acidosis or liver failure. Many of those who survive develop signs and symptoms later in life of a related disorder called Kearns-Sayre syndrome. This condition causes weakness of muscles around the eyes and other problems.

### Undiagnosed Cholestatic Pruritus

Undiagnosed cholestatic pruritus refers to pruritus that is associated with cholestasis, but the underlying cause of the cholestasis has not yet been identified.

### ASBT Inhibitors

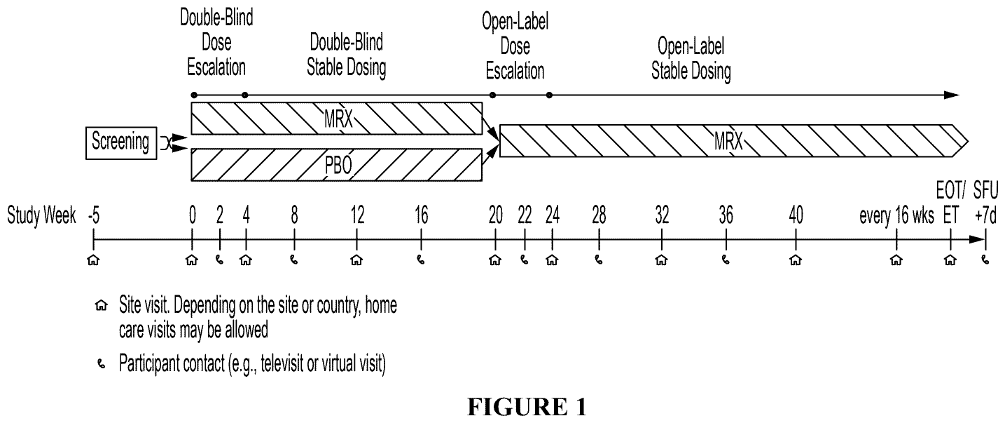

In various embodiments of methods of the present invention, an ASBT inhibitor is administered to a subject. In some embodiments, the ASBTI inhibitor is maralixibat, or a pharmaceutically acceptable salt thereof. In some embodiments, the ASBTI inhibitor is maralixibat chloride. ASBT inhibitors (ASBTIs) reduce or inhibit bile acid recycling in the distal gastrointestinal (GI) tract, including the distal ileum, the colon and/or the rectum. Inhibition of the apical sodium-dependent bile acid transport interrupts the enterohepatic circulation of bile acids and results in more bile acids being excreted in the feces, see FIG. 1, leading to lower levels of bile acids systemically, thereby reducing bile acid mediated liver damage and related effects and complications. In certain embodiments, the ASBTIs are systemically absorbed. In certain embodiments, the ASBTIs are not systemically absorbed. In one embodiment, maralixibat is a non-systemically absorbed ASBTI. In some embodiments, the ASBTI used in the methods or compositions of the present invention is

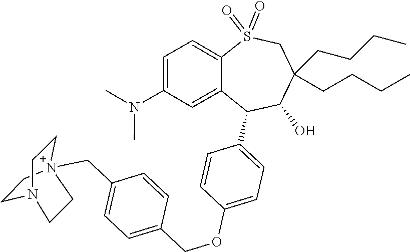 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O]

maralixibat, or a pharmaceutically acceptable salt thereof.

In one embodiment, the ASBTI used in the methods or compositions of the present invention is

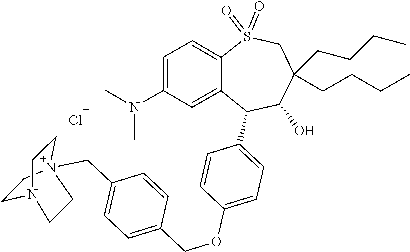 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

(maralixibat chloride, LUM-001, SHP625, lopixibat chloride).

In one embodiment, the ASBTI used in the methods or compositions of the present invention is

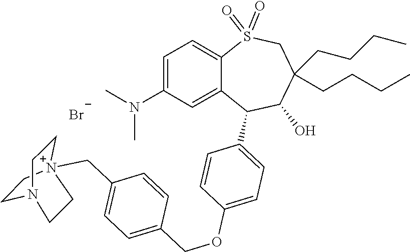 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Br-]]

maralixibat bromide.

In one embodiment, the ASBTI used in the methods or compositions of the present invention is

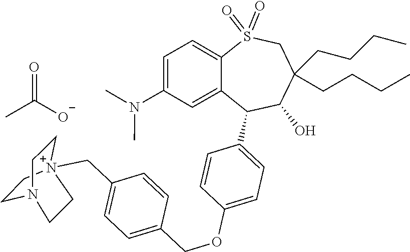

maralixibat acetate.

In one embodiment, the ASBTI used in the methods or compositions of the present invention is

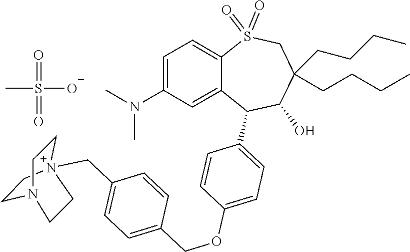

maralixibat mesylate.

### Methods for Treating Cholestasis

Provided herein is a method for treating cholestasis in a subject having a liver disease. The method comprises administering to a subject in need of treatment an Apical Sodium-dependent Bile Acid Transporter Inhibitor (ASBTI). The ASBTI is maralixibat, maralixibat chloride, or an alternative pharmaceutically acceptable salt thereof. The ASBTI is administered in an amount of from about 300 μg/kg/day to about 1200 μg/kg/day.

In various embodiments, the liver disease is a cholestatic liver disease. In one embodiment, the cholestatic liver disease is a rare cholestatic liver disease.

In one embodiment, the rare cholestatic liver disease is selected from the group consisting of biliary atresia (BA), Caroli's disease, Caroli's syndrome, ciliopathies (Joubert syndrome, Meckel-Gruber syndrome, NPHP3 mutation), alpha-1-antitrypsin deficiency, chronic idiopathic hepatitis, chronic viral hepatitis, secondary sclerosing cholangitis (related to COVID-19 or injury), IgG4-related sclerosing cholangitis, ARC, Langerhans cell histiocytosis, sodium taurocholate co-transporting polypeptide deficiency, transaldolase deficiency, X-linked myotubular myopathy, hepatic sarcoidosis, idiopathic amyloidosis, ischemic cholangiopathy, rare metabolic disorders, nonalcoholic fatty liver disease, post-liver transplant cholestasis (including patients with ALGS, PBC, PFIC, or PSC who have had liver transplant), and undiagnosed cholestatic pruritus.

In one embodiment, the rare cholestatic liver disease is selected from the group consisting of Caroli's disease, ciliopathies (Joubert syndrome, Meckel-Gruber syndrome, NPHP3 mutation), alpha-1-antitrypsin deficiency, chronic idiopathic hepatitis, secondary sclerosing cholangitis (related to COVID-19 or injury), IgG4-related sclerosing cholangitis, ARC, Langerhans cell histiocytosis, sodium taurocholate co-transporting polypeptide deficiency, transaldolase deficiency, X-linked myotubular myopathy, hepatic sarcoidosis, idiopathic amyloidosis, ischemic cholangiopathy, rare metabolic disorders, post-liver transplant cholestasis (including patients with ALGS, PBC, PFIC, or PSC who have had liver transplant), and undiagnosed cholestatic pruritus.

In one embodiment, the rare cholestatic liver disease is not ALGS, PBC, PFIC, or PSC. In one embodiment, the rare cholestatic liver disease is not BA. In one embodiment, the rare cholestatic liver disease is not nonalcoholic fatty liver disease.

In certain embodiments, the cholestatic liver disease is characterized by one or more symptoms selected from jaundice, pruritus, cirrhosis, hypercholemia, neonatal respiratory distress syndrome, lung pneumonia, increased serum concentration of bile acids, increased hepatic concentration of bile acids, increased serum concentration of bilirubin, hepatocellular injury, liver scarring, liver failure, hepatomegaly, xanthomas, malabsorption, splenomegaly, diarrhea, pancreatitis, hepatocellular necrosis, giant cell formation, hepatocellular carcinoma, gastrointestinal bleeding, portal hypertension, hearing loss, fatigue, loss of appetite, anorexia, peculiar smell, dark urine, light stools, steatorrhea, failure to thrive, and/or renal failure.

In some embodiments, the treatment comprises reducing one or more of cholestatic pruritus, serum bile acids, and bilirubin.

In one embodiment, the treatment reduces cholestatic pruritus.

In one embodiment, the treatment reduces sBA.

In one embodiment, the treatment reduces bilirubin.

In one aspect, the present disclosure provides a method for treating cholestatic pruritus in a subject having a cholestatic liver disease. The method comprises administering to a subject in need of treatment an Apical Sodium-dependent Bile Acid Transporter Inhibitor (ASBTI). The ASBTI is maralixibat, maralixibat chloride, or an alternative pharmaceutically acceptable salt thereof. The ASBTI is administered in an amount of from about 300 μg/kg/day to about 1200 μg/kg/day.

In some embodiments, the subject is a pediatric subject. In various embodiments, the patient is a pediatric patient under the age of 1 month, 2 months, 3 months, 4 months, 5 months, 6 months, 7 months, 8 months, 9 months, 10 months, 11 months, or 1, 2, 3, 4, 5, 6, 7, 8, 9, 10, 11, 12, 13, 14, 15, 16, 17, or 18 years old. In certain embodiments, the pediatric subject is a newborn, a pre-term newborn, an infant, a toddler, a preschooler, a school-age child, a pre-pubescent child, post-pubescent child, an adolescent, or a teenager under the age of eighteen. In some embodiments, the pediatric subject is a newborn, a pre-term newborn, an infant, a toddler, a preschooler, or a school-age child. In some embodiments, the pediatric subject is a newborn, a pre-term newborn, an infant, a toddler, or a preschooler. In some embodiments, the pediatric subject is a newborn, a pre-term newborn, an infant, or a toddler. In some embodiments, the pediatric subject is a newborn, a pre-term newborn, or an infant. In some embodiments, the pediatric subject is a newborn. In some embodiments, the pediatric subject is an infant. In some embodiments, the pediatric subject is a toddler.

In some embodiments, the subject is an adult. In some embodiments, the subject is over 18 years of age, or over 25 years of age, or over 50 years of age, or over 60 years of age, or over 65 years of age, or over 70 years of age, or over 75 years of age. In certain embodiments, methods of the present invention comprise non-systemic administration of a therapeutically effective amount of maralixibat or maralixibat chloride. In certain embodiments, the methods comprise contacting the gastrointestinal tract, including the distal ileum and/or the colon and/or the rectum, of an individual in need thereof with maralixibat or maralixibat chloride. In various embodiments, the methods of the present invention cause a reduction in intraenterocyte bile acids, or a reduction in damage to hepatocellular or intestinal architecture caused by cholestasis or a cholestatic liver disease.

In various embodiments, methods of the present invention comprise delivering to ileum or colon of the individual a therapeutically effective amount of maralixibat or maralixibat chloride.

In various embodiments, methods of the present invention comprise reducing damage to hepatocellular or intestinal architecture or cells from cholestasis or a cholestatic liver disease comprising administration of a therapeutically effective amount of maralixibat or maralixibat chloride. In certain embodiments, the methods of the present invention comprise reducing intraenterocyte bile acids/salts through administration of a therapeutically effective amount of maralixibat or maralixibat chloride to an individual in need thereof.

In some embodiments, methods of the present invention provide for inhibition of bile salt recycling upon administration of any of the compounds described herein to an individual. In some embodiments, maralixibat or maralixibat chloride is not absorbed systemically. In some embodiments, maralixibat or maralixibat chloride is administered to the individual orally. In some embodiments, maralixibat or maralixibat chloride is delivered and/or released in the distal ileum of an individual.

In various embodiments, contacting the distal ileum of an individual with an ASBTI (e.g., maralixibat or maralixibat chloride) inhibits bile acid reuptake and increases the concentration of bile acids/salts in the vicinity of L-cells in the distal ileum and/or colon and/or rectum, thereby reducing intraenterocyte bile acids, reducing serum and/or hepatic bile acid levels, reducing overall serum bile acid load, and/or reducing damage to ileal architecture caused by cholestasis or a cholestatic liver disease. Without being limited to any particular theory, reducing serum and/or hepatic bile acid levels ameliorates hypercholemia and/or cholestatic disease.

Administration of a compound described herein may be achieved in any suitable manner including, by way of non-limiting example, by oral, enteric, parenteral (e.g., intravenous, subcutaneous, intramuscular), intranasal, buccal, topical, rectal, or transdermal administration routes. Any compound or composition described herein may be administered in a method or formulation appropriate to treat a newborn or an infant. Any compound or composition described herein may be administered in an oral formulation (e.g., solid or liquid) to treat a newborn or an infant. Any compound or composition described herein may be administered prior to ingestion of food, with food or after ingestion of food.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, the individual's health status, weight, and response to the drugs, and the judgment of the treating physician.

In prophylactic applications, compounds or compositions containing compounds described herein may be administered to an individual susceptible to or otherwise at risk of a particular disease, disorder or condition. In certain embodiments of this use, the precise amounts of compound administered depend on the individual's state of health, weight, and the like. Furthermore, in some instances, when a compound or composition described herein is administered to an individual, effective amounts for this use depend on the severity and course of the disease, disorder or condition, previous therapy, the individual's health status and response to the drugs, and the judgment of the treating physician.

In certain embodiments of the methods of the present invention, wherein following administration of a selected dose of a compound or composition described herein, an individual's condition does not improve, upon the doctor's discretion the administration of a compound or composition described herein is optionally administered chronically, that is, for an extended period of time, including throughout the duration of the individual's life in order to ameliorate or otherwise control or limit the symptoms of the individual's disorder, disease or condition.

In certain embodiments of the methods of the present invention, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In some embodiments, doses administered include those up to the maximum tolerable dose by a newborn or an infant.

In various embodiments of the methods of the present invention, a desired dose is conveniently presented in a single dose or in divided doses administered simultaneously (or over a short period of time) or at appropriate intervals, for example as two, three, four or more sub-doses per day. In various embodiments, a single dose of maralixibat or maralixibat chloride is administered every 6 hours, every 12 hours, every 24 hours, every 48 hours, every 72 hours, every 96 hours, every 5 days, every 6 days, or once a week. In some embodiments the total single dose of maralixibat or maralixibat chloride is in a range described below.

In various embodiments of methods of the present invention, in the case wherein the patient's status does improve, upon the doctor's discretion maralixibat or maralixibat chloride is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100% of the original dose, including, by way of example only, 10%, 15%, 20%, 25%, 30%, 35%, 40%, 45%, 50%, 55%, 60%, 65%, 70%, 75%, 80%, 85%, 90%, 95%, or 100% of the original dose. In some embodiments the total single dose of an ASBTI is in a range described below.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

### Dosages

In various embodiments the ASBTI is maralixibat, or a pharmaceutically acceptable salt thereof.

In various embodiments, efficacy and safety of ASBTI administration to the patient is monitored by measuring serum levels of 7α-hydroxy-4-cholesten-3-one (7αC4), sBA concentration, a ratio of 7αC4 to sBA (7αC4:sBA), serum conjugated bilirubin concentration, serum autotaxin concentration, serum fibroblast growth factor (FGF-19) concentration, serum bilirubin concentration, serum total cholesterol concentration, serum LDL-C concentration, serum ALT concentration, serum AST concentration, or a combination thereof. In various embodiments, efficacy of ASBTI administration is measured by monitoring observer-reported itch reported outcome (ITCHRO (OBS)) score, a HRQoL (e.g., PedsQL) score, a CSS score, a xanthoma score, a height Z-score, a weight Z-score, or various combinations thereof. In various embodiments, the method includes monitoring serum levels of 7αC4, sBA concentration, a ratio of 7αC4 to sBA (7αC4:sBA), serum conjugated bilirubin concentration, serum total cholesterol concentration, serum LDL-C concentration, serum autotaxin concentration, serum bilirubin concentration, serum ALT concentration, serum AST concentration, or a combination thereof. In various embodiments, the method includes monitoring observer-reported itch reported outcome (ITCHRO (OBS)) score, a weight Z-score, a HRQoL (e.g., PedsQL) score, a xanthoma score, a CSS score, a height Z-score, or various combinations thereof.

In some embodiments, the ASBTI is administered at a dose of about or at least about 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 200 μg/kg, 240 μg/kg, 280 μg/kg, 300 μg/kg, 250 μg/kg, 280 μg/kg, 300 μg/kg, 400 μg/kg, 500 μg/kg, 560 μg/kg, 600 μg/kg, 700 μg/kg, 800 μg/kg, 900 μg/kg, 1,000 μg/kg, 1,100 μg/kg, 1,200 μg/kg, 1,300 μg/kg, 1,400 μg/kg, 1500 μg/kg, 1,600 μg/kg, 1,700 μg/kg, 1,800 μg/kg, 1,900 μg/kg, or 2,000 μg/kg. In various embodiments, the ASBTI is administered at a dose not exceeding about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 100 μg/kg, 140 μg/kg, 150 μg/kg, 200 μg/kg, 240 μg/kg, 280 μg/kg, 300 μg/kg, 250 μg/kg, 280 μg/kg, 300 μg/kg, 400 μg/kg, 500 μg/kg, 560 μg/kg, 600 μg/kg, 700 μg/kg, 800 μg/kg, 900 μg/kg, 1,000 μg/kg, 1,100 μg/kg, 1,200 μg/kg, 1,300 μg/kg, 1,400 μg/kg, 1,500 μg/kg, 1,600 μg/kg, 1,700 μg/kg, 1,800 μg/kg, 1,900 μg/kg, 2,000, or 2,100 μg/kg. In various embodiments, the ASBTI is administered at a dose of about or of at least about 0.5 mg/day, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, 1000 mg/day. In various embodiments, the ASBTI is administered at a dose of not more than about 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 30 mg/day, 40 mg/day, 50 mg/day, 60 mg/day, 70 mg/day, 80 mg/day, 90 mg/day, 100 mg/day, 150 mg/day, 200 mg/day, 300 mg/day, 500 mg/day, 600 mg/day, 700 mg/day, 800 mg/day, 900 mg/day, 1,000 mg/day, 1,100 mg/day.

In some embodiments, maralixibat is administered at a dose of from about 140 μg/kg/day to about 1400 μg/kg/day. In various embodiments, maralixibat is administered at a dose of about or at least about 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 200 μg/kg/day, 240 μg/kg/day, 250 μg/kg/day, 280 μg/kg/day, 300 μg/kg/day, 400 μg/kg/day, 500 μg/kg/day, 560 μg/kg/day, 600 μg/kg/day, 700 μg/kg/day, 800 μg/kg/day, 900 μg/kg/day, 1,000 μg/kg/day, 1,100 μg/kg/day, 1,200 μg/kg/day, or 1,300 μg/kg/day. In various embodiments, maralixibat is administered at a dose not exceeding about 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day 10 μg/kg/day, 15 μg/kg/day, 20 μg/kg/day, 25 μg/kg/day, 30 μg/kg/day, 35 μg/kg/day, 40 μg/kg/day, 45 μg/kg/day, 50 μg/kg/day, 100 μg/kg/day, 140 μg/kg/day, 150 μg/kg/day, 200 μg/kg/day, 240 μg/kg/day, 280 μg/kg/day, 300 μg/kg/day, 250 μg/kg/day, 280 μg/kg/day, 300 μg/kg/day, 400 μg/kg/day, 500 μg/kg/day, 560 μg/kg/day, 600 μg/kg/day, 700 μg/kg/day, 800 μg/kg/day, 900 μg/kg/day, 1,000 μg/kg/day, 1,100 μg/kg/day, 1,200 μg/kg/day, 1,300 μg/kg/day, or 1,400 μg/kg/day. In various embodiments, maralixibat is administered at a dose of from about 0.5 μg/kg/day to about 500 μg/kg/day, from about 0.5 μg/kg/day to about 250 μg/kg/day, from about 1 μg/kg/day to about 100 μg/kg/day, from about 10 μg/kg/day to about 50 μg/kg/day, from about 10 μg/kg/day to about 100 μg/kg/day, from about 0.5 μg/kg/day to about 2000 μg/kg/day, from about 280 μg/kg/day to about 1400 μg/kg/day, from about 420 μg/kg/day to about 1400 μg/kg/day, from about 250 to about 550 μg/kg/day, from about 560 μg/kg/day to about 1400 μg/kg/day, from 700 μg/kg/day to about 1400 μg/kg/day, from about 560 μg/kg/day to about 1200 μg/kg/day, from about 700 μg/kg/day to about 1200 μg/kg/day, from about 560 μg/kg/day to about 1000 μg/kg/day, from about 700 μg/kg/day to about 1000 μg/kg/day, from about 800 μg/kg/day to about 1000 μg/kg/day, from about 200 μg/kg/day to about 600 μg/kg/day, from about 300 μg/kg/day to about 600 μg/kg/day, from about 400 μg/kg/day to about 500 μg/kg/day, from about 400 μg/kg/day to about 600 μg/kg/day, from about 400 μg/kg/day to about 700 μg/kg/day, from about 400 μg/kg/day to about 800 μg/kg/day, from about 500 μg/kg/day to about 800 μg/kg/day, from about 500 μg/kg/day to about 900 μg/kg/day, from about 600 μg/kg/day to about 900 μg/kg/day, from about 700 μg/kg/day to about 900 μg/kg/day, from about 200 μg/kg/day to about 600 μg/kg/day, from about 800 μg/kg/day to about 900 μg/kg/day, from about 100 μg/kg/day to about 1500 μg/kg/day, from about 300 μg/kg/day to about 2,000 μg/kg/day, or from about 400 μg/kg/day to about 2000 μg/kg/day.

In some embodiments, maralixibat is administered at a dose of from about 30 μg/kg to about 1400 μg/kg per dose. In some embodiments, maralixibat is administered at a dose of from about 0.5 μg/kg to about 2000 μg/kg per dose, from about 0.5 μg/kg to about 1500 μg/kg per dose, from about 100 μg/kg to about 700 μg/kg per dose, from about 5 μg/kg to about 100 μg/kg per dose, from about 10 μg/kg to about 500 μg/kg per dose, from about 50 μg/kg to about 1400 μg/kg per dose, from about 300 μg/kg to about 2,000 μg/kg per dose, from about 60 μg/kg to about 1200 μg/kg per dose, from about 70 μg/kg to about 1000 μg/kg per dose, from about 70 μg/kg to about 700 μg/kg per dose, from 80 μg/kg to about 1000 μg/kg per dose, from 80 μg/kg to about 800 μg/kg per dose, from 100 μg/kg to about 800 μg/kg per dose, from 100 μg/kg to about 600 μg/kg per dose, from 150 μg/kg to about 700 μg/kg per dose, from 150 μg/kg to about 500 μg/kg per dose, from 200 μg/kg to about 400 μg/kg per dose, from 200 μg/kg to about 300 μg/kg per dose, or from 300 μg/kg to about 400 μg/kg per dose, or from 400 μg/kg to about 800 μg/kg per dose, or from 500 μg/kg to about 700 μg/kg per dose, or from 5500 μg/kg to about 650 μg/kg per dose.

In some embodiments, maralixibat is administered at a dose of from about 0.5 mg/day to about 550 mg/day. In various embodiments, maralixibat is administered at a dose of from about 1 mg/day to about 500 mg/day, from about 1 mg/day to about 300 mg/day, from about 1 mg/day to about 200 mg/day, from about 2 mg/day to about 300 mg/day, from about 2 mg/day to about 200 mg/day, from about 4 mg/day to about 300 mg/day, from about 4 mg/day to about 200 mg/day, from about 4 mg/day to about 150 mg/day, from about 5 mg/day to about 150 mg/day, from about 5 mg/day to about 100 mg/day, from about 5 mg/day to about 80 mg/day, from about 5 mg/day to about 50 mg/day, from about 5 mg/day to about 40 mg/day, from about 5 mg/day to about 30 mg/day, from about 5 mg/day to about 20 mg/day, from about 5 mg/day to about 15 mg/day, from about 10 mg/day to about 100 mg/day, from about 10 mg/day to about 80 mg/day, from about 10 mg/day to about 50 mg/day, from about 10 mg/day to about 40 mg/day, from about 10 mg/day to about 20 mg/day, from about 20 mg/day to about 100 mg/day, from about 20 mg/day to about 80 mg/day, from about 20 mg/day to about 50 mg/day, or from about 20 mg/day to about 40 mg/day, or from about 20 mg/day to about 30 mg/day, or from about 30 mg/day to about 80 mg/day, or from about 40 mg/day to about 70 mg/day, or from about 50 mg/day to about 60 mg/day.

In some embodiments, maralixibat is administered twice daily (BID) in an amount of about 150 μg/kg to about 600 μg/kg per dose. In some embodiments, maralixibat is administered in an amount of about 280 μg/kg/day to about 1400 μg/kg/day. In some embodiments, maralixibat is administered in an amount of about 400 μg/kg/day to about 800 μg/kg/day. In some embodiments, maralixibat is administered in an amount of about 10 mg/day to about 100 mg/day. In some embodiments, maralixibat is administered in an amount of from about 15 mg/day to about 60 mg/day. In some embodiments, maralixibat is administered in an amount of from about 560 μg/kg/day to about 1,200 μg/kg/day. In some embodiments, maralixibat is administered in an amount of from about 700 μg/kg/day to about 1,400 μg/kg/day. In some embodiments, maralixibat is administered in an amount of from about 400 μg/kg/day to about 800 μg/kg/day. In some embodiments, maralixibat is administered in an amount of from about 700 μg/kg/day to about 900 μg/kg/day. In some embodiments, maralixibat is administered in an amount of from about 560 μg/kg/day to about 1400 μg/kg/day. In some embodiments, maralixibat is administered in an amount from 700 μg/kg/day to about 1400 μg/kg/day. In some embodiments, maralixibat is administered in an amount of from about 200 μg/kg/day to about 600 μg/kg/day. In some embodiments, maralixibat is administered in an amount of from about 400 μg/kg/day to about 600 μg/kg/day. In some embodiments, maralixibat is administered in an amount of from about 1100 μg/kg/day to about 1200 μg/kg/day. In some embodiments, maralixibat is administered in an amount of about 570 μg/kg/day twice daily (BID) of maralixibat, based on maralixibat free base, which is equivalent to an amount of about 600 μg/kg/day twice daily (BID) of maralixibat chloride.

In various embodiments, the dose of maralixibat is a first dose level. In various embodiments, the dose of maralixibat is a second dose level. In various embodiments, the dose of maralixibat is a third dose level. In various embodiments, the dose of maralixibat is a fourth dose level. In some embodiments, the second dose level is greater than the first dose level. In some embodiments, the second dose level is about or at least about 1.5, 2, 3, 4, 5, 6, 7, 8, 9, 10, 15, 20, 30, 40, 50, 60, 70, 80, 90 or 100 times or fold greater than the first dose level. In some embodiments, the second dose level is not in excess of about 1.5, 2, 3, 4, 5, 6, 7, 8, 9, 10, 15, 20, 30, 40, 50, 60, 70, 80, 90, 100, or 150 times or fold greater than the first dose level. In some embodiments, the third dose level is greater than the second dose level. In some embodiments, the third dose level is about or at least about 1.5, 2, 3, 4, 5, 6, 7, 8, 9, 10, 15, 20, 30, 40, 50, 60, 70, 80, 90 or 100 times or fold greater than the second dose level. In some embodiments, the third dose level is not in excess of about 1.5, 2, 3, 4, 5, 6, 7, 8, 9, 10, 15, 20, 30, 40, 50, 60, 70, 80, 90, 100, or 150 times or fold greater than the second dose level. In some embodiments, the fourth dose level is greater than the third dose level. In some embodiments, the fourth dose level is about or at least about 1.5, 2, 3, 4, 5, 6, 7, 8, 9, 10, 15, 20, 30, 40, 50, 60, 70, 80, 90 or 100 times or fold greater than the third dose level. In some embodiments, the fourth dose level is not in excess of about 1.5, 2, 3, 4, 5, 6, 7, 8, 9, 10, 15, 20, 30, 40, 50, 60, 70, 80, 90, 100, or 150 times or fold greater than the third dose level.

In various embodiments, maralixibat is administered once daily (QD) at one of the above doses or within one of the above dose ranges. In various embodiments, maralixibat is administered twice daily (BID) at one of the above doses or within one of the above dose ranges. In various embodiments, an ASBTI dose is administered daily, every other day, twice a week, or once a week.

In various embodiments, maralixibat is administered regularly for a period of about or of at least about 1, 2, 3, 4, 5, 6, 7, 8, 9, 10, 15, 20, 30, 40, 48, 50, 75, 100, 150, 200, 250, 300, 350, 400, 450, 500, 600, 700, or 800 weeks. In various embodiments, maralixibat is administered for not more than about 1, 2, 3, 4, 5, 6, 7, 8, 9, 10, 15, 20, 30, 40, 48, 50, 75, 100, 150, 200, 250, 300, 350, 400, 450, 500, 600, 700, 800, or 1000 weeks. In various embodiments, maralixibat is administered regularly for a period of about or of at least about 0.5, 1, 1.5, 2, 3, 4, 5, 6, 7, 8, 9, or 10 years. In various embodiments, maralixibat is administered regularly for a period not in excess of about 0.5, 1, 1.5, 2, 3, 4, 5, 6, 7, 8, 9, 10, or 15 years.

### Reduction in Symptoms or a Change in a Disease-Relevant Laboratory Measures of Cholestatic Liver Disease

In various embodiments of the above methods of the invention, administration of maralixibat or maralixibat chloride results in a reduction in a symptom or a change in a disease-relevant laboratory measure of the cholestatic liver disease (i.e., improvement in the patient's condition) that is maintained for about or for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years. In various embodiments, the reduction in the symptom or a change in a disease-relevant laboratory measure comprises a reduction in sBA concentration, an increase in serum 7αC4 concentration, an increase in the 7αC4:sBA ratio, an increase in fBA excretion, a reduction in pruritus, a decrease in serum total cholesterol concentration, a decrease in serum LDL-C cholesterol concentration, a reduction in ALT levels, an increase in a quality of life inventory score, an increase in a quality of life inventory score related to fatigue, a reduction in a xanthoma score, a reduction in serum autotaxin concentration, an increase in growth, or a combination thereof. In various embodiments, the reduction in the symptom or a change in a disease-relevant laboratory measure comprises a reduction in sBA concentration, a reduction in pruritus, a decrease in total bilirubin, a decrease in direct bilirubin, an increase in growth, or a combination thereof. In various embodiments, the reduction in the symptom or a change in a disease-relevant laboratory measure is determined relative to a baseline level. That is, the reduction in the symptom or a change in a disease-relevant laboratory measure is determined relative to a measurement of the symptom or a change in a disease-relevant laboratory measure prior to 1) changing a dose level of the ASBTI administered to the patient, 2) changing a dosing regimen followed for the patient, 3) commencing administration of the ASBTI, or 4) any other of various alterations made with the intention of reducing the symptom or a change in a disease-relevant laboratory measure in the patient. In various embodiments, the reduction in symptom or a change in a disease-relevant laboratory measure is a statistically significant reduction.

In various embodiments, the reduction in a symptom or a change in a disease-relevant laboratory measure of the cholestatic liver disease is measured as a progressive decrease in the symptom or a change in a disease-relevant laboratory measure for about or for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years.

In some embodiments, the patient is the pediatric patient and the reduction in symptom or a change in a disease-relevant laboratory measure comprises an increase or improvement in growth. In some embodiments, the increase in growth is measured relative to baseline. In various embodiments, increase in growth is measured as an increase in height Z-score or in weight Z-score. In various embodiments, the increase in height Z-score or in weight Z-score is statistically significant. In various embodiments, the height Z-score, the weight Z-score, or both is increased by at least 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29 0.3, 0.31, 0.32, 0.33, 0.34, 0.35, 0.36, 0.37, 0.38, 0.39 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.7, 0.8, or 0.9 relative to baseline. In some embodiments, the height Z-score, the weight Z-score, or both progressively increases during administration of the ASBTI for a period of about or of at least about 1, 2, 3, 4, 5, 6, 7, 8, 9, 10, 20, 30, 40, 48, 50, 60, 70, or 72 weeks.

In various embodiments, the administration of the ASBTI results in an increase in serum 7αC4 concentration. In various embodiments, the serum 7αC4 concentration is increased by about or at least about 1.5, 2, 3, 4, 5, 6, 7, 8, 9, 10, 15, 20, 30, 40, 50, 60, 70, 80, 90, 100, 200, 300, 400, or 500 times or fold relative to baseline. In various embodiments the serum 7αC4 concentration is increased about or at least about 10%, 20%, 30%, 40%, 50%, 60%, 70%, 80%, 90%, 100%, 150%, 200%, 300%, 400%, 500%, 600%, 700%, 800%, 900%, 1,000%, or 10,000% relative to baseline.

In various embodiments, the administration of the ASBTI results in decrease of total bilirubin. In various embodiments, the total bilirubin is decreased by about or at least about 0.1, 0.2, 0.3, 0.4, 0.5, 0.6, 0.7, 0.8, or 0.9 times or fold relative to baseline. In various embodiments the total bilirubin is decreased by about or at least about 10%, 20%, 30%, 40%, 50%, 60%, 70%, 80%, 90%, or 100% relative to baseline. In various embodiments the total bilirubin is decreased by about or at least about 0.1 mg/dL, 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1.0 mg/dL, 1.1 mg/dL, or 1.2 mg/dL relative to baseline.

In various embodiments, the administration of the ASBTI results in decrease of direct bilirubin. In various embodiments, the direct bilirubin is decreased by about or at least about 0.1, 0.2, 0.3, 0.4, 0.5, 0.6, 0.7, 0.8, or 0.9 times or fold relative to baseline. In various embodiments the direct bilirubin is decreased by about or at least about 10%, 20%, 30%, 40%, 50%, 60%, 70%, 80%, 90%, or 100 relative to baseline. In various embodiments the direct bilirubin is decreased by about or at least about 0.1 mg/dL, 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1.0 mg/dL, 1.1 mg/dL, or 1.2 mg/dL relative to baseline.

In various embodiments, the administration of the ASBTI results in an increase in the 7αC4:sBA ratio to about or by at least about 1.25, 1.5, 1.75, 2, 2.5, 3, 4, 5, 6, 7, 8, 9, 10, 15, 20, 30, 40, 50, 75, 100, 150, 200, 300, 500, 750, 1,000, 2,000, 3,000, 4,000, 5,000 or 10,000-fold relative to baseline.

In various embodiments, the administration of the ASBTI results in an increase in fBA excretion. In some embodiments, the administration of the ASBTI results in an increase in fBA excretion of about or of at least about 100%, 110%, 115%, 120%, 130%, 150%, 200%, 250%, 275%, 300%, 400%, 500%, 600%, 700%, 800%, 1,000%, 5,000%, 10,000% or 15,000% relative to baseline. In various embodiments, fBA excretion is increased by about or by at least about 1, 1.5, 2, 3, 4, 5, 6, 7, 8, 9, 10, 20, 30, 40, 50, 60, 70, 80, 90, or 100 fold or times relative to baseline. In some embodiments, fBA excretion is increased by about or by at least about 100 μmol, 150 μmol, 200 μmol, 250 μmol, 300 μmol, 400 μmol, 500 μmol, 600 μmol, 700 μmol, 800 μmol, 900 μmol, 1,000 μmol, or 1,500 μmol relative to baseline. In various embodiments, administration of the ASBTI results in a dose-dependent increase in fBA excretion so that administration of a higher dose of the ASBTI results in a corresponding higher level of fBA excretion. In various embodiments, the ASBTI is administered at a dose sufficient to result in an increase in bile acid secretion relative to baseline of at least about or of about 1, 2, 3, 4, 5, 6, 7, 8, 9, 10, 20, 30, 40, 50, 60, 70, 80, 90, or 100 fold or times relative to baseline.

In various embodiments, the administration of the ASBTI results in a decrease in sBA concentration of about or of at least about 5%, 10%, 15%, 20%, 25%, 30%, 31%, 35%, 40%, 45%, 50%, 55%, 57%, 60%, 65%, 70%, 75%, 80%, 85%, 90%, or 95% relative to baseline.

In some embodiments, the administration of the ASBTI results in a reduction in severity of pruritus. In various embodiments, the severity of pruritus is measured using an ITCHRO (OBS) score, an ITCHRO score, a CSS score, or a combination thereof. In various embodiments, the administration of the ASBTI results in a reduction in the ITCHRO (OBS) score on a scale of 1 to 4 of about or of at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, or 3 relative to baseline. In various embodiments, the administration of the ASBTI results in a reduction in the ITCHRO score on a scale of 1 to 10 of about or of at least about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, or 10. In various embodiments, the administration of the ASBTI results in a reduction of the ITCHRO (OBS) score, the ITCHRO score, or both to zero. In various embodiments, the administration of the ASBTI results in a reduction of the ITCHRO (OBS) score or ITCHRO score to 1.0 or lower. In various embodiments, the administration of the ASBTI results in a reduction of the CSS score by about of at least about 0.1, 0.2, 0.3, 0.4, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, or 3 relative to baseline. In various embodiments, the administration of the ASBTI results in a reduction of the CSS score to zero. In various embodiments, the administration of the ASBTI results in a reduction in the CSS score, the ITCHRO (OBS) score, the ITCHRO score, or a combination thereof by about or by at least about 10%, 15%, 20%, 25%, 30%, 35%, 40%, 45%, 50%, 55%, 60%, 65%, 70%, 75%, 80%, 85%, 90%, 95%, or 100% relative to baseline. In various embodiments, a reduced value relative to baseline of the CSS score, the ITCHRO (OBS) score, the ITCHRO score, or a combination thereof is observed on 10%, 20%, 30%, 40%, 50%, 60%, 70%, 75%, 80%, 85%, 90%, 91%, 92%, 93%, 94%, 95%, 96%, 97%, 98%, 99%, or 100% of days.

In some embodiments, patients with a higher baseline ITCHRO (OBS) score demonstrate a greater reduction in the symptom or a change in a disease-relevant laboratory measure than patients having a lower baseline ITCHRO (OBS) score. In some embodiments, patients with a baseline ITCHRO (OBS) score of at least 2, 3, or 4 or an ITCHRO score of at least 4, 5, 6, 7, 8, 9, or 10 have a greater reduction in the symptom or a change in a disease-relevant laboratory measure relative to baseline than a lower reduction in patients having a lower baseline severity of pruritus score. In various embodiments, patients having PSC and baseline ITCHRO scores of at least 4 demonstrate a greater reduction in the symptom or a change in a disease-relevant laboratory measure than patients having a baseline ITCHRO score of less than 4. In various embodiments, the method includes predicting that a patient will have a greater reduction in the symptom or a change in a disease-relevant laboratory measure if a baseline ITCHRO score of the patient is at least 4 as compared to a patient having a baseline ITCHRO score of less than 4. In various embodiments the lower reduction is about or less than about 5%, 10%, 15%, 20%, 25%, 30%, 35%, 40%, 45%, 50%, 55%, or 60% the greater reduction. In various embodiments a difference in the reduction in the symptom or a change in a disease-relevant laboratory measure (i.e., between the greater reduction and the lower reduction) between patients having an ITCHRO score of at least 4 at baseline and patients having an ITCHRO score of less than 4 at baseline is measured at about or at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 6 months, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 13 months, 14 months, 15 months, 16 months, 17 months, 18 months, 19 months, 20 months, 21 months, 22 months, 23 months, 23 months, 2 years, 2.5 years, 3 years, 3.5 years, 4 years, 4.5 years, 5 years, 5.5 years, 6 years, 6.5 years, 7 years, 8 years, 9 years, or 10 years following first administration of the ASBTI at the first dose or at the second dose.

In various embodiments, reduction in severity of pruritus caused by administration of the ASBTI to the patient is positively correlated with a reduction in sBA concentration in the patient. In various embodiments, a greater reduction in sBA concentration in the patient correlates with a corresponding greater reduction in severity of pruritus.

In various embodiments, the administration of the ASBTI results in a reduction in serum LDL-C concentration relative to baseline. In some embodiments the serum LDL-C concentration is reduced by about or by at least about 1%, 2%, 3%, 4%, 5%, 10%, 15%, 20%, 25%, 25%, 30%, 35%, 40%, 45%, 50%, 55%, 60%, 65%, 70%, 75%, or 80% relative to baseline.

In some embodiments, the administration of the ASBTI results in a reduction in serum total cholesterol concentration relative to baseline. In some embodiments, the administration of the ASBTI results in a reduction in serum LDL-C levels relative to baseline. In some embodiments the serum total cholesterol concentration, the serum LDL-C levels, or both is reduced by about or by at least about 1%, 2%, 3%, 4%, 5%, 10%, 15%, 20%, 25%, 25%, 30%, 35%, 40%, 45%, 50%, 55%, 60%, 65%, 70%, 75%, or 80% relative to baseline. In various embodiments, the administration of the ASBTI results in a reduction in serum total cholesterol concentration, of serum LDL-C levels, or both of about or of at least about 1 mg/dL, 2 mg/dL, 3 mg/dL, 4 mg/dL, 5 mg/dL, 10 mg/dL, 12.5 mg/dL, 15 mg/dL, 20 mg/dL, 30 mg/dL, 40 mg/dL or 50 mg/dL relative to baseline.

In various embodiments, the administration of the ASBTI results in a decrease in serum autotaxin concentration. In some embodiments, the administration of the ASBTI results in a reduction in autotaxin concentration of about or of at least about 5%, 10%, 15%, 20%, 25%, 30%, 35%, 40%, 45%, 50%, 55%, 60%, 65%, 70%, 75%, or 80% relative to baseline.

In various embodiments, the administration of the ASBTI results in improvements to sleep. In some embodiments, the sleep is assessed using Exploratory Diary Questionnaire (EDQ(Obs)). In some embodiments the average morning EDQ(Obs) sleep disturbance scores are decreased by about or by at least about 1%, 2%, 3%, 4%, 5%, 10%, 15%, 20%, 25%, 25%, 30%, 35%, 40%, 45%, 50%, 55%, 60%, 65%, 70%, 75%, or 80% relative to baseline.

In some embodiments, the administration of the ASBTI results in an improvement in sleep as measured by a reduction of an EDQ(Obs) or EDQ(Pt) score of the subject by at least 1.0 points relative to baseline. In some embodiments, the administration of the ASBTI results in an improvement in sleep as measured by a reduction of an EDQ(Obs) or EDQ(Pt) score of the subject by at least 1.2 points relative to baseline. In some embodiments, the administration of the ASBTI results in an improvement in sleep as measured by a reduction of an EDQ(Obs) or EDQ(Pt) score of the subject by at least 1.4 points relative to baseline. In some embodiments, the administration of the ASBTI results in an improvement in sleep as measured by a reduction of an EDQ(Obs) or EDQ(Pt) score of the subject by at least 1.6 points relative to baseline.

In various embodiments, administration of the ASBTI results in an increase in a quality of life inventory score or in a quality of life inventory score related to fatigue. The quality of life inventory score can be a health-related quality of life (HRQoL) score. In some embodiments, the HRQoL score is a PedsQL score. In various embodiments, the administration of the ASBTI results an increase in the PedsQL score or in a PedsQL score related to fatigue of about or of at least about 5%, 10%, 15%, 20%, 25%, 30%, 45%, or 50% relative to baseline.

In various embodiments, administration of the ASBTI results in a decrease in a xanthoma score relative to baseline. In some embodiments, the xanthoma score is reduced by about or by at least about 2.5%, 5%, 10%, 15%, 20%, 35%, 30%, 35%, 40%, 45%, 50%, 55%, 60%, 65%, 70%, 75%, 80%, 85%, 90%, 95%, or 100% relative to baseline.

In various embodiments, the administration of the ASBTI results in the reduction in the symptom or a change in a disease-relevant laboratory measure by about 1 day, 2 days, 3 days, 4 days, 5 days, 6 days, 7 days, 8 days, 9 days, 10 days, 11 days, 12, days, 13 days, 14 days, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year.

In various embodiments, serum bilirubin concentration is at pre-administration baseline levels or at normal levels at about or by about 1 day, 2 days, 3 days, 4 days, 5 days, 6 days, 1 week, 2 weeks, 3 weeks, 4 weeks, 1 month, 5 weeks, 6 weeks, 7 weeks, 8 weeks, 2 months, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year.

In various embodiments, serum ALT concentration is at pre-administration baseline levels or at normal levels at about or by about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year. In some embodiments, the administration of the ASBTI results in a reduction in ALT levels relative to baseline of about or of at least about 1%, 2%, 3%, 4%, 5%, 6%, 7%, 8%, 9%, 10%, 11%, 12%, 13%, 14%, or 15%.

In various embodiments, serum ALT concentration, serum AST concentration, serum bilirubin concentration, serum conjugated bilirubin concentration, or various combinations thereof are within normal range or at pre-administration baseline levels at about or by about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year. In various embodiments, the administration of the ASBTI does not result in a statistically significant change from baseline in serum bilirubin concentration, serum AST concentration, serum ALT concentration, serum alkaline phosphatase concentration, or some combination thereof for a period of at least about or of about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year. In various embodiments, for adult patients with an ITCHRO score of at least 4 at baseline, the administration of the ASBTI does not result in a significant change from baseline in serum conjugated bilirubin concentration for a period of at least about or of about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks 10 weeks, 11 weeks, 12 weeks, 13 weeks, 14 weeks, 15 weeks, 16 weeks, 4 months, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or 1 year.

### Dose Modulation

In various embodiments, the method includes modulating a dosage of maralixibat administered to the patient. In some embodiments, modulating a dosage of maralixibat may comprise escalating the dosage of maralixibat. In some embodiments, the modulation includes administering maralixibat at a first dose level to the patient for a first week. If the patient tolerates the first dose level, the dose level is increased to a second dose level for a second week. If the patient tolerates the second dose level, the dose level is increased to a third dose level for a third week. If the patient tolerates the third dose level, the dose level is increased to a fourth dose level for the remainder of the treatment study.

In some embodiments, the method comprises a Dose Escalation period. In one non-limiting embodiment, the Dose Escalation period comprises the following weekly steps: a) Dose level 1:300 μg/kg (or 15 mg) maralixibat QD for 1 week; b) Dose level 2:300 μg/kg (or 15 mg) maralixibat BID for 1 week; d) Dose level 3:600 μg/kg (or 30 mg) maralixibat BID for the remaining duration of the administration. In another non-limiting embodiment, dose escalation steps may be delayed or reversed to improve tolerability.

### Pharmaceutical Compositions

In some embodiments, maralixibat is administered as a pharmaceutical composition comprising maralixibat or maralixibat chloride). Any composition described herein can be formulated for ileal, rectal and/or colonic delivery. In more specific embodiments, the composition is formulated for non-systemic or local delivery to the rectum and/or colon. It is to be understood that, as used herein, delivery to the colon includes delivery to sigmoid colon, transverse colon, and/or ascending colon. In still more specific embodiments, the composition is formulated for non-systemic or local delivery to the rectum and/or colon is administered rectally. In other specific embodiments, the composition is formulated for non-systemic or local delivery to the rectum and/or colon is administered orally.

Provided herein, in certain embodiments, is a pharmaceutical composition comprising a therapeutically effective amount of any compound described herein. In certain instances, the pharmaceutical composition comprises an ASBT inhibitor (e.g., maralixibat or maralixibat chloride).

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Mareel Decker, New York, N.Y., 1980; and Pharmaceutical Dosage Forms and Drug Delivery Systems, Seventh Ed. (Lippincott Williams & Wilkins1999), all of which references are incorporated herein in their entirety for all purposes.

A pharmaceutical composition, as used herein, refers to a mixture of a compound described herein, with other chemical components, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the compounds described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes.

In certain embodiments, a pharmaceutical compositions described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” includes, in some embodiments, a pharmaceutically acceptable excipient and is selected on the basis of compatibility with compounds described herein, such as, compounds of any of Formula I-VI, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Mareel Decker, New York, N.Y., 1980; and Pharmaceutical Dosage Forms and Drug Delivery Systems, Seventh Ed. (Lippincott Williams & Wilkins 1999), all of which references are incorporated herein in their entirety for all purposes.

Moreover, in certain embodiments, the pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

### Routes of Administration, Dosage Forms, and Dosing Regimens

In some embodiments, the compositions described herein, and the compositions administered in the methods described herein are formulated to inhibit bile acid reuptake or reduce serum or hepatic bile acid levels. In certain embodiments, the compositions described herein are formulated for rectal or oral administration. In some embodiments, such formulations are administered rectally or orally, respectively. In some embodiments, the compositions described herein are combined with a device for local delivery of the compositions to the rectum and/or colon (sigmoid colon, transverse colon, or ascending colon). In certain embodiments, for rectal administration the composition described herein are formulated as enemas, rectal gels, rectal foams, rectal aerosols, suppositories, jelly suppositories, or retention enemas. In some embodiments, for oral administration the compositions described herein are formulated for oral administration and enteric delivery to the colon.

In certain embodiments, the compositions or methods described herein are non-systemic. In some embodiments, compositions described herein deliver the ASBTI to the distal ileum, colon, and/or rectum and not systemically (e.g., a substantial portion of the enteroendocrine peptide secretion enhancing agent is not systemically absorbed). In some embodiments, oral compositions described herein deliver the ASBTI to the distal ileum, colon, and/or rectum and not systemically (e.g., a substantial portion of the enteroendocrine peptide secretion enhancing agent is not systemically absorbed). In some embodiments, rectal compositions described herein deliver the ASBTI to the distal ileum, colon, and/or rectum and not systemically (e.g., a substantial portion of the enteroendocrine peptide secretion enhancing agent is not systemically absorbed). In certain embodiments, non-systemic compositions described herein deliver less than 90% w/w of the ASBTI systemically. In certain embodiments, non-systemic compositions described herein deliver less than 80% w/w of the ASBTI systemically. In certain embodiments, non-systemic compositions described herein deliver less than 70% w/w of the ASBTI systemically. In certain embodiments, non-systemic compositions described herein deliver less than 60% w/w of the ASBT1 systemically. In certain embodiments, non-systemic compositions described herein deliver less than 50% w/w of the ASBTI systemically. In certain embodiments, non-systemic compositions described herein deliver less than 40% w/w of the ASBTI systemically. In certain embodiments, non-systemic compositions described herein deliver less than 30% w/w of the ASBTI systemically. In certain embodiments, non-systemic compositions described herein deliver less than 25% w/w of the ASBTI systemically. In certain embodiments, non-systemic compositions described herein deliver less than 20% w/w of the ASBTI systemically. In certain embodiments, non-systemic compositions described herein deliver less than 15% w/w of the ASBTI systemically. In certain embodiments, non-systemic compositions described herein deliver less than 10% w/w of the ASBTI systemically. In certain embodiments, non-systemic compositions described herein deliver less than 5% w/w of the ASBTI systemically. In some embodiments, systemic absorption is determined in any suitable manner, including the total circulating amount, the amount cleared after administration, or the like.

In certain embodiments, the compositions and/or formulations described herein are administered at least once a day. In certain embodiments, the formulations containing the ASBTI are administered at least twice a day, while in other embodiments the formulations containing the ASBTI are administered at least three times a day. In certain embodiments, the formulations containing the ASBTI are administered up to five times a day. It is to be understood that in certain embodiments, the dosage regimen of composition containing the ASBTI described herein to is determined by considering various factors such as the patient's age, sex, and diet.

The concentration of the ASBTI administered in the formulations described herein ranges from about 1 mM to about 1 M. In certain embodiments the concentration of the ASBTI administered in the formulations described herein ranges from about 1 mM to about 750 mM. In certain embodiments the concentration of the ASBTI administered in the formulations described herein ranges from about 1 mM to about 500 mM. In certain embodiments the concentration of the ASBTI administered in the formulations described herein ranges from about 5 mM to about 500 mM. In certain embodiments the concentration of the ASBTI administered in the formulations described herein ranges from about 10 mM to about 500 mM. In certain embodiments the concentration of the administered in the formulations described herein ranges from about 25 mM to about 500 mM. In certain embodiments the concentration of the ASBTI administered in the formulations described herein ranges from about 50 mM to about 500 mM. In certain embodiments the concentration of the ASBTI administered in the formulations described herein ranges from about 100 mM to about 500 mM. In certain embodiments the concentration of the ASBTI administered in the formulations described herein ranges from about 200 mM to about 500 mM.

In certain embodiments, by targeting the distal gastrointestinal tract (e.g., distal ileum, colon, and/or rectum), compositions and methods described herein provide efficacy (e.g., in reducing microbial growth and/or alleviating symptoms of cholestasis or a cholestatic liver disease) with a reduced dose of enteroendocrine peptide secretion enhancing agent (e.g., as compared to an oral dose that does not target the distal gastrointestinal tract).

### Liquid Dosage Forms

The pharmaceutical liquid dosage forms of the invention may be prepared according to techniques well-known in the art of pharmacy.

A solution refers to a liquid pharmaceutical formulation wherein the active ingredient is dissolved in the liquid. Pharmaceutical solutions of the invention include syrups and elixirs. A suspension refers to a liquid pharmaceutical formulation wherein the active ingredient is in a precipitate in the liquid.

In a liquid dosage form, it is desirable to have a particular pH and/or to be maintained within a specific pH range. In order to control the pH, a suitable buffer system can be used. In addition, the buffer system should have sufficient capacity to maintain the desired pH range. Examples of the buffer system useful in the present invention include but are not limited to, citrate buffers, phosphate buffers, or any other suitable buffer known in the art. Preferably the buffer system include sodium citrate, potassium citrate, sodium bicarbonate, potassium bicarbonate, sodium dihydrogen phosphate and potassium dihydrogen phosphate, etc. The concentration of the buffer system in the final suspension varies according to factors such as the strength of the buffer system and the pH/pH ranges required for the liquid dosage form. In one embodiment, the concentration is within the range of 0.005 to 0.5 w/v % in the final liquid dosage form.

The pharmaceutical composition comprising the liquid dosage form of the present invention can also include a suspending/stabilizing agent to prevent settling of the active material. Over time the settling could lead to caking of the active to the inside walls of the product pack, leading to difficulties with redispersion and accurate dispensing. Suitable stabilizing agents include but are not limited to, the polysaccharide stabilizers such as xanthan, guar and tragacanth gums as well as the cellulose derivatives HPMC (hydroxypropyl methylcellulose), methyl cellulose and Avicel RC-591 (microcrystalline cellulose/sodium carboxymethyl cellulose). In another embodiment, polyvinylpyrrolidone (PVP) can also be used as a stabilizing agent.

In addition to the aforementioned components, the ASBTI oral suspension form can also optionally contain other excipients commonly found in pharmaceutical compositions such as alternative solvents, taste-masking agents, antioxidants, fillers, acidifiers, enzyme inhibitors and other components as described in Handbook of Pharmaceutical Excipients, Rowe et al., Eds., 4th Edition, Pharmaceutical Press (2003), which is hereby incorporated by reference in its entirety for all purposes.

Addition of an alternative solvent may help increase solubility of an active ingredient in the liquid dosage form, and consequently the absorption and bioavailability inside the body of a subject. Preferably the alternative solvents include methanol, ethanol or propylene glycol and the like.

In another aspect, the present invention provides a process for preparing the liquid dosage form. The process comprises steps of bringing maralixibat or its pharmaceutically acceptable salts thereof into mixture with the components including glycerol or syrup or the mixture thereof, a preservative, a buffer system and a suspending/stabilizing agent, etc., in a liquid medium. In general, the liquid dosage form is prepared by uniformly and intimately mixing these various components in the liquid medium. For example, the components such as glycerol or syrup or the mixture thereof, a preservative, a buffer system and a suspending/stabilizing agent, etc., can be dissolved in water to form the aqueous solution, then the active ingredient can be then dispersed in the aqueous solution to form a suspension.

In some embodiments, the liquid dosage form provided herein can be in a volume of between about 5 ml to about 50 ml. In some embodiments, the liquid dosage form provided herein can be in a volume of between about 5 ml to about 40 ml. In some embodiments, the liquid dosage form provided herein can be in a volume of between about 5 ml to about 30 ml. In some embodiments, the liquid dosage form provided herein can be in a volume of between about 5 ml to about 20 ml. In some embodiments, the liquid dosage form provided herein can be in a volume of between about 10 ml to about 30 ml. In some embodiments, the liquid dosage form provided herein can be in a volume of about 20 ml. In some embodiments, maralixibat can be in an amount ranging from about 0.001% to about 90% of the total volume. In some embodiments, maralixibat can be in an amount ranging from about 0.01% to about 80% of the total volume. In some embodiments, maralixibat can be in an amount ranging from about 0.1% to about 70% of the total volume. In some embodiments, maralixibat can be in an amount ranging from about 1% to about 60% of the total volume. In some embodiments, maralixibat can be in an amount ranging from about 1% to about 50% of the total volume. In some embodiments, maralixibat can be in an amount ranging from about 1% to about 40% of the total volume. In some embodiments, maralixibat can be in an amount ranging from about 1% to about 30% of the total volume. In some embodiments, maralixibat can be in an amount ranging from about 1% to about 20% of the total volume. In some embodiments, maralixibat can be in an amount ranging from about 1% to about 10% of the total volume. In some embodiments, maralixibat can be in an amount ranging from about 5% to about 70% of the total volume. In some embodiments, maralixibat can be in an amount ranging from about 5% to about 60% of the total volume. In some embodiments, maralixibat can be in an amount ranging from about 5% to about 50% of the total volume. In some embodiments, maralixibat can be in an amount ranging from about 5% to about 40% of the total volume. In some embodiments, maralixibat can be in an amount ranging from about 5% to about 30% of the total volume. In some embodiments, maralixibat can be in an amount ranging from about 5% to about 20% of the total volume. In some embodiments, maralixibat can be in an amount ranging from about 5% to about 10% of the total volume. In some embodiments, maralixibat can be in an amount ranging from about 10% to about 50% of the total volume. In some embodiments, maralixibat can be in an amount ranging from about 10% to about 40% of the total volume. In some embodiments, maralixibat can be in an amount ranging from about 10% to about 30% of the total volume. In some embodiments, maralixibat can be in an amount ranging from about 10% to about 20% of the total volume. In one embodiment, the resulted liquid dosage form can be in a liquid volume of 10 ml to 30 ml, preferably 20 ml, and the active ingredient can be in an amount ranging from about 0.001 mg/ml to about 25 mg/ml, or from about 0.025 mg/ml to about 8 mg/ml, or from about 0.1 mg/ml to about 4 mg/ml, or about 0.25 mg/ml, or about 0.5 mg/ml, or about 1 mg/ml, or about 2 mg/ml, or about 4 mg/ml, or about 5 mg/ml, or about 8 mg/ml, or about 10 mg/ml, or about 12 mg/ml, or about 14 mg/ml or about 16 mg/ml, or about 18 mg/ml, or about 20 mg/ml, or about 25 mg/ml. In one embodiment, the active ingredient is maralixibat present in an amount of 9.5 mg/ml. In one embodiment, the active ingredient is maralixibat chloride present in an amount of 10 mg/ml.

### Oral Solution

In some embodiments, the pharmaceutical composition is formulated as an oral solution comprising maralixibat chloride, a preservative, an antioxidant, a flavoring agent, a sweetener, and water.

### Preservative

In certain embodiments, the compositions of the present invention comprise a preservative. In certain embodiments, the preservative is an antimicrobial preservative.

In certain embodiments, the antimicrobial preservative is selected from the group consisting of propylene glycol, ethyl alcohol, glycerin, benzalkonium chloride, benzethonium chloride, benzoic acid, benzyl alcohol, butylparaben, cetrimide (cetyltrimethylammonium bromide), cetrimonium bromide, cetylpyridinium chloride, chlorhexidine, chlorobutanol, chlorocresol, cresol, ethylparaben, methylparaben, phenol, phenoxyethanol, phenylethyl alcohol, phenylmercuric acetate, phenylmercuric borate, phenylmercuric nitrate, propylparaben, sodium benzoate, sodium dehydroacetate, sodium propionate, sorbic acid, potassium sorbate, thimerosal, thymol, and combinations thereof.

In certain embodiments, the preservative is propylene glycol.

In certain embodiments, the preservative is present in an amount of at least about 10% w/w of the composition. In certain embodiments, the preservative is present in an amount of at least about 20% w/w of the composition. In certain embodiments, the preservative is present in an amount of at least about 25% w/w of the composition. In certain embodiments, the preservative is present in an amount of at least about 30% w/w of the composition.

In certain embodiments, the preservative is present in an amount of from about 30% to about 40% of the composition.

In certain embodiments, the preservative is present in an amount of from about 32% to about 37% of the composition. In certain embodiments, the preservative is present in an amount of from about 33% to about 36% of the composition.

In certain embodiments, the preservative is present in an amount of about 33% of the composition. In certain embodiments, the preservative is present in an amount of about 34% of the composition. In certain embodiments, the preservative is present in an amount of about 35% of the composition.

### Antioxidant

In certain embodiments, the compositions of the present invention comprise an antioxidant. In certain embodiments, the antioxidant is selected from the group consisting of an aminocarboxylic acid, an aminopolycarboxylic acid, ascorbic acid, ascorbyl palmitate, butylated hydroxyanisole, butylated hydroxytoluene, monothioglycerol, sodium ascorbate, sodium formaldehyde sulfoxylate, sodium metabisulfite, BHT, BHA, sodium bisulfite, vitamin E or a derivative thereof, propyl gallate, and combinations thereof.

In certain embodiments, the antioxidant is an aminopolycarboxylic acid selected from EDTA (ethylenediaminetetraacetic acid), DTPA (diethylenetriaminepentaacetic acid), EGTA (ethylene glycol-bis(β-aminoethyl ether)-N,N,N′,N′-tetraacetic acid), NTA (nitrilotriacetic acid), BAPTA (1,2-bis(o-aminophenoxy) ethane-N,N,N′,N′-tetraacetic acid), NOTA (2,2′,2″-(1,4,7-triazonane-1,4,7-triyl)triacetic acid), DOTA (tetracarboxylic acid), and EDDHA (ethylenediamine-N,N′-bis(2-hydroxyphenylacetic acid)

In certain embodiments, the antioxidant is EDTA.

In certain embodiments, the antioxidant is present in an amount of about 0.001% to about 1% w/w of the composition. In certain embodiments, the antioxidant is present in an amount of about 0.005% to about 0.75% w/w of the composition. In certain embodiments, the antioxidant is present in an amount of about 0.01% to about 0.5% w/w of the composition. In certain embodiments, the antioxidant is present in an amount of about 0.05% to about 0.25% w/w of the composition. In certain embodiments, the antioxidant is present in an amount of about 0.075% to about 0.2% w/w of the composition. In certain embodiments, the antioxidant is present in an amount of about 0.1% w/w of the composition.

In some embodiments, the pharmaceutical composition comprises from about 5 mg/mL to about 50 mg/mL of maralixibat chloride; from about 300 mg/mL to about 400 mg/mL of propylene glycol; about 1 mg/mL of disodium EDTA; a sweetener, a flavoring agent, or a combination thereof, and water.

In some embodiments, the pharmaceutical composition comprises from about 5 mg/mL to about 50 mg/mL of maralixibat chloride; from about 300 mg/mL to about 400 mg/mL of propylene glycol; about 1 mg/mL of disodium EDTA; about 10 mg/mL sucralose, about 5 mg/mL grape flavor, and water.

In some embodiments, the pharmaceutical composition comprises about 10 mg/mL of maralixibat chloride; about 360 mg/mL of propylene glycol; about 1 mg/mL of disodium EDTA; about 10 mg/mL sucralose, about 5 mg/mL grape flavor, and water.

### Pediatric Dosage Formulations and Compositions

Provided herein, in certain embodiments, is a pediatric dosage formulation or composition comprising a therapeutically effective amount of any compound described herein. In certain instances, the pharmaceutical composition comprises an ASBT inhibitor (e.g., maralixibat or maralixibat chloride).

In certain embodiments, suitable dosage forms for the pediatric dosage formulation or composition include, by way of non-limiting example, aqueous or non-aqueous oral dispersions, liquids, gels, syrups, elixirs, slurries, suspensions, solutions, controlled release formulations, fast melt formulations, effervescent formulations, lyophilized formulations, chewable tablets, gummy candy, orally disintegrating tablets, powders for reconstitution as suspension or solution, sprinkle oral powder or granules, dragees, delayed release formulations, extended release formulations, pulsatile release formulations, multiparticulate formulations, and mixed immediate release and controlled release formulations. In some embodiments, provided herein is a pharmaceutical composition wherein the pediatric dosage form is selected from a solution, syrup, suspension, elixir, powder for reconstitution as suspension or solution, dispersible/effervescent tablet, chewable tablet, gummy candy, lollipop, freezer pops, troches, oral thin strips, orally disintegrating tablet, orally disintegrating strip, sachet, and sprinkle oral powder or granules.

In another aspect, provide herein is a pharmaceutical composition wherein at least one excipient is a flavoring agent or a sweetener. In some embodiments, provided herein is a coating. In some embodiments, provided herein is a taste-masking technology selected from coating of drug particles with a taste-neutral polymer by spray-drying, wet granulation, fluidized bed, and microencapsulation; coating with molten waxes of a mixture of molten waxes and other pharmaceutical adjuvants; entrapment of drug particles by complexation, flocculation or coagulation of an aqueous polymeric dispersion; adsorption of drug particles on resin and inorganic supports; and solid dispersion wherein a drug and one or more taste neutral compounds are melted and cooled, or co-precipitated by a solvent evaporation. In some embodiments, provided herein is a delayed or sustained release formulation comprising drug particles or granules in a rate controlling polymer or matrix.

Suitable sweeteners include sucrose, glucose, fructose or intense sweeteners, i.e. agents with a high sweetening power when compared to sucrose (e.g. at least 10 times sweeter than sucrose). Suitable intense sweeteners comprise aspartame, saccharin, sodium or potassium or calcium saccharin, acesulfame potassium, sucralose, alitame, xylitol, cyclamate, neomate, neohesperidine dihydrochalcone or mixtures thereof, thaumatin, palatinit, stevioside, rebaudioside, Magnasweet®. The total concentration of the sweeteners may range from effectively zero to about 300 mg/ml based on the liquid composition upon reconstitution.

In order to increase the palatability of the liquid composition upon reconstitution with an aqueous medium, one or more taste-making agents may be added to the composition in order to mask the taste of the ASBT inhibitor. A taste-masking agent can be a sweetener, a flavoring agent or a combination thereof. The taste-masking agents typically provide up to about 0.1% or 5% by weight of the total pharmaceutical composition. In a preferred embodiment of the present invention, the composition contains both sweetener(s) and flavor(s).

A flavoring agent herein is a substance capable of enhancing taste or aroma of a composition. Suitable natural or synthetic flavoring agents can be selected from standard reference books, for example Fenaroli's Handbook of Flavor Ingredients, 3rd edition (1995). Non-limiting examples of flavoring agents and/or sweeteners useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. Flavoring agents can be used singly or in combinations of two or more. In some embodiments, the aqueous liquid dispersion comprises a sweetening agent or flavoring agent in a concentration ranging from about 0.001% to about 5.0% the volume of the aqueous dispersion. In one embodiment, the aqueous liquid dispersion comprises a sweetening agent or flavoring agent in a concentration ranging from about 0.001% to about 1.0% the volume of the aqueous dispersion. In another embodiment, the aqueous liquid dispersion comprises a sweetening agent or flavoring agent in a concentration ranging from about 0.005% to about 0.5% the volume of the aqueous dispersion. In yet another embodiment, the aqueous liquid dispersion comprises a sweetening agent or flavoring agent in a concentration ranging from about 0.01% to about 1.0% the volume of the aqueous dispersion. In yet another embodiment, the aqueous liquid dispersion comprises a sweetening agent or flavoring agent in a concentration ranging from about 0.01% to about 0.5% the volume of the aqueous dispersion.

In certain embodiments, a pediatric pharmaceutical composition described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” for pediatric pharmaceutical compositions includes, in some embodiments, a pharmaceutically acceptable excipient and is selected on the basis of compatibility with compounds described herein, such as, compounds of any of Formula I-VI, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), all of which references are incorporated herein by reference in their entirety for all purposes.

Moreover, in certain embodiments, the pediatric pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In certain aspects, the pediatric composition or formulation containing one or more compounds described herein is orally administered for local delivery of maralixibat, maralixibat chloride, or other compounds described herein to the colon and/or rectum. Unit dosage forms of such compositions include a pill, tablet or capsules formulated for enteric delivery to colon. In certain embodiments, such pills, tablets or capsule contain the compositions described herein entrapped or embedded in microspheres. In some embodiments, microspheres include, by way of non-limiting example, chitosan microcores HPMC capsules and cellulose acetate butyrate (CAB) microspheres. In certain embodiments, oral dosage forms are prepared using conventional methods known to those in the field of pharmaceutical formulation. For example, in certain embodiments, tablets are manufactured using standard tablet processing procedures and equipment. An exemplary method for forming tablets is by direct compression of a powdered, crystalline or granular composition containing the active agent(s), alone or in combination with one or more carriers, additives, or the like. In alternative embodiments, tablets are prepared using wet-granulation or dry-granulation processes. In some embodiments, tablets are molded rather than compressed, starting with a moist or otherwise tractable material.

In certain embodiments, tablets prepared for oral administration contain various excipients, including, by way of non-limiting example, binders, diluents, lubricants, disintegrants, fillers, stabilizers, surfactants, preservatives, coloring agents, flavoring agents and the like. In some embodiments, binders are used to impart cohesive qualities to a tablet, ensuring that the tablet remains intact after compression. Suitable binder materials include, by way of non-limiting example, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), Veegum, and combinations thereof. In certain embodiments, diluents are utilized to increase the bulk of the tablet so that a practical size tablet is provided. Suitable diluents include, by way of non-limiting example, dicalcium phosphate, calcium sulfate, lactose, cellulose, kaolin, mannitol, sodium chloride, dry starch, powdered sugar and combinations thereof. In certain embodiments, lubricants are used to facilitate tablet manufacture; examples of suitable lubricants include, by way of non-limiting example, vegetable oils such as peanut oil, cottonseed oil, sesame oil, olive oil, corn oil, and oil of theobroma, glycerin, magnesium stearate, calcium stearate, stearic acid and combinations thereof. In some embodiments, disintegrants are used to facilitate disintegration of the tablet, and include, by way of non-limiting example, starches, clays, celluloses, algins, gums, crosslinked polymers and combinations thereof. Fillers include, by way of non-limiting example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. In certain embodiments, stabilizers are used to inhibit or retard drug decomposition reactions that include, by way of example, oxidative reactions. In certain embodiments, surfactants are anionic, cationic, amphoteric or nonionic surface active agents.

In some embodiments, maralixibat, maralixibat chloride, or other compounds described herein are orally administered in association with a carrier suitable for delivery to the distal gastrointestinal tract (e.g., distal ileum, colon, and/or rectum).

In certain embodiments, a pediatric composition described herein comprises maralixibat, maralixibat chloride, or other compounds described herein in association with a matrix (e.g., a matrix comprising hypermellose) that allows for controlled release of an active agent in the distal part of the ileum and/or the colon. In some embodiments, a composition comprises a polymer that is pH sensitive (e.g., a MMX™ matrix from Cosmo Pharmaceuticals) and allows for controlled release of an active agent in the distal part of the ileum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO₃H) and swell in basic pH of the intestine (e.g., pH of about 7 to about 8). In some embodiments, a composition suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an enteroendocrine peptide secretion enhancing agent to the distal ileum. In some embodiments, a dosage form comprising an enteroendocrine peptide secretion enhancing agent is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the distal ileum and/or the colon. In some embodiments, bacterially activated systems are suitable for targeted delivery to the distal part of the ileum. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

The pediatric pharmaceutical composition described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of Formula I. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

In further embodiments, a tablet or capsule comprising an ASBTI or other compounds described herein is film-coated for delivery to targeted sites within the gastrointestinal tract. Examples of enteric film coats include and are not limited to hydroxypropylmethylcellulose, polyvinyl pyrrolidone, hydroxypropyl cellulose, polyethylene glycol 3350, 4500, 8000, methyl cellulose, pseudo ethylcellulose, amylopectin and the like.

### Solid Dosage Forms for Pediatric Administration

Solid dosage forms for pediatric administration of the present invention can be manufactured by standard manufacturing techniques. Non-limiting examples of oral solid dosage forms for pediatric administration are described below.

### Effervescent Compositions

The effervescent compositions of the invention may be prepared according to techniques well-known in the art of pharmacy.

Effervescent formulations contain and effervescent couple of a base component and an acid component, which components reach in the presence of water to generate a gas. In some embodiments, the base component may comprise, for example, an alkali metal or alkaline earth metal carbonate, or bicarbonate. The acid component may comprise, for example, an aliphatic carboxylic acid or a salt thereof, such as citric acid. The base and acid components may each independently constitute, for example, 25% to 55% (w/w) of the effervescent composition. The ratio of acid component to base component may be within the range of 1:2 to 2:1.

The effervescent compositions of the invention may be formulated using additional pharmaceutically acceptable carriers or excipients as appropriate. For example, one or more taste masking agents may be used. Dyes may also be used, as pediatric patients often prefer colorful pharmaceutical combinations. The compositions may take the form of, for example, tablets, granules or powders, granules or powders presented in a sachet.

### Chewable Tablets

The chewable tablets of the invention may be prepared according to techniques well-known in the art of pharmacy.

Chewable tablets are tablets that are intended to disintegrate in the mouth under the action of chewing or sucking and where, in consequence, the active ingredient has greater opportunity to come into contact with the bitter-taste receptors on the tongue.

One method of overcoming this issue is to absorb the active ingredient onto a suitable substrate. This approach is known in the art and described for example in U.S. Pat. No. 4,647,459, which is incorporated herein by reference in its entirety for all purposes.

Another approach involves forming the active ingredient into an aggregate along with a pre-swelled substantially anhydrous hydrocolloid. The hydrocolloid absorbs saliva and acquires a slippery texture which enables it to lubricate the particles of aggregate and mask the taste of the active ingredient. This approach is known in the art and described for example in European patent application 0190826, which is incorporated herein by reference in its entirety for all purposes.

Another approach involves employing a water-insoluble hygroscopic excipient such as microcrystalline cellulose. This approach is known in the art and described for example in U.S. Pat. No. 5,275,823, which is incorporated herein by reference in its entirety for all purposes.

In addition to the above approaches, the chewable tablets of the present invention can also contain other standard tableting excipients such as a disintegrant and a taste-masking agent.

### Orodispersible Tablets

The orodispersible tablets of the invention may be prepared according to techniques well-known in the art of pharmacy.

In orodispersible tablets of the invention, the excipient mixtures are such as to provide it with a disintegration rate so that its disintegration in the buccal cavity occurs in an extremely short time and especially shorter than sixty seconds. In some embodiments, the excipient mixture is characterized by the fact that the active substance is in the form of coated or non-coated microcrystals of microgranules. In some embodiments, the orodispersible tablet comprises one or several disintegrating agents of the carboxymethylcellulose type or insoluble reticulated PVP type, one or several swelling agents which may comprise a carboxymethylcellulose, a starch, a modified starch, or a microcrystalline cellulose or optionally a direct compression sugar.

### Powders for Reconstitution

The powder for reconstitution pharmaceutical compositions of the invention may be prepared according to techniques well-known in the art of pharmacy.

In some embodiments, the powder for reconstitution compositions of the invention comprise an effective amount of at least one internal dehydrating agent. The internal dehydrating agent can enhance the stability of the powder. In some embodiments, the internal dehydrating agent is magnesium citrate or disodium carbonate. In some embodiments, the powder composition comprises a pharmaceutically acceptable diluents, such as sucrose, dextrose, mannitol, xylitol, or lactose.

Powder compositions of the inventions may be placed in sachets or bottles for contemporaneous dissolution or for short term storage in liquid form (e.g. 7 days).

### Gummy Candies

The gummy candies of the invention may be prepared according to techniques well-known in the art of pharmacy.

Traditional gummy candy is made from a gelatin base. Gelatin gives the candy its elasticity, the desired chewy consistency, and a longer shelf life. In some embodiments, the gummy candy pharmaceutical composition of the invention includes a binding agent, a sweetener, and an active ingredient.

In some embodiments, the binding agent is a pectin gel, gelatin, food starch, or any combination thereof.

In some embodiments, the gummy candy comprises sweeteners, a binding agent, natural and/or artificial flavors and colors and preservatives. In some embodiments, the gummy candy comprises glucose syrup, natural cane juice, gelatin, citric acid, lactic acid, natural colors, natural flavors, fractionated coconut oil, and carnauba wax.

An ASBT inhibitor (e.g., maralixibat) may be used in the preparation of medicaments for the prophylactic and/or therapeutic treatment of cholestasis or a cholestatic liver disease (e.g., PFIC). A method for treating any of the diseases or conditions described herein in an individual in need of such treatment, may involve administration of pharmaceutical compositions containing at least one ASBT inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

### EXAMPLES

The following examples illustrate specific aspects of the instant description. The examples should not be construed as limiting, as the examples merely provide specific understanding and practice of the embodiments and their various aspects.

### Abbreviations

Abbreviations are defined in Table 1.

| Abbreviation | Definition |
|---|---|
| 7αC4 | alpha-hydroxy-4-cholesten-3-one |
| β-hCG | beta-human chorionic gonadotropin |
| ADL | activities of daily living |
| AE | adverse event |
| ALGS | Alagille syndrome |
| APRI | AST-to-platelet ratio index |
| ARC | arthrogryposis with renal dysfunction and cholestasis |
| BA | biliary atresia |
| BID | twice daily |
| BMI | body mass index |
| CFR | Code of Federal Regulations |
| CIC | Caregiver Impression of Change |
| CIS | Caregiver Impression of Severity |
| CSS | Clinician Scratch Scale |
| CTCAE | Common Terminology Criteria for Adverse Events |
| DCT | decentralized clinical trial |
| eCRF | electronic case report form |
| EAIR | exposure adjusted incidence rate |
| EOT | end of treatment |
| ET | early termination |
| FDA | US Food and Drug Administration |
| FGF-19 | fibroblast growth factor-19 |
| FSH | follicle stimulating hormone |
| FSV | fat-soluble vitamin |
| GCP | Good Clinical Practice |
| HPE | hepatoportoenterostomy |
| IBAT | ileal bile acid transporter |
| IB | Investigator's Brochure |
| ICF | informed consent form |
| ICH | International Council for Harmonisation of Technical |
| | Requirements for Pharmaceuticals for Human Use |
| ICP | intrahepatic cholestasis of pregnancy |
| IEC | Institutional Ethics Committee |
| Ig | immunoglobulin |
| INR | international normalized ratio |
| IP | investigational product |
| IRB | Institutional Review Board |
| ItchRO ™ | Itch-Reported Outcome |
| ItchRO(Obs) ™ | Observer-Reported Itch-Reported Outcome Instrument |
| ItchRO(Pt) ™ | Patient-Reported Itch-Reported Outcome Instrument |
| IRT | interactive response technology |
| ITT | intent-to-treat (population) |
| MMRM | mixed effect model for repeated measures |
| MRI | magnetic resonance imaging |
| PBC | primary biliary cholangitis |
| PFIC | progressive familial intrahepatic cholestasis |
| PIC | Patient Impression of Change |
| PIS | Patient Impression of Severity |
| PRO | patient-reported outcome |
| PSC | primary sclerosing cholangitis |
| QD | once daily (dosing) |
| QoL | quality of life |
| SAE | serious adverse event |
| SAP | statistical analysis plan |
| SBA | serum bile acid |
| SoA | Schedule of Activities |
| SOC | System Organ Class |
| TEAE | treatment-emergent AE |
| VAP | validation analysis plan |
| WOCBP | woman of childbearing potential |

### Example 1: Clinical Study Design

The study is an international, multicenter, randomized, double-blind, placebo-controlled study followed by open-label dosing.

### Screening

All participants who consent to the study will undergo the screening period for a minimum of 2 weeks (maximum of 5 weeks) prior to being eligible for study enrollment. During the screening period, participants or their caregivers will document their cholestatic itch severity by daily completion of the morning ItchRO(Obs)/ItchRO(Pt) severity questionnaire (Item 1) to establish eligibility and a baseline score. Participants who meet all of the inclusion and none of the exclusion criteria will enter the study.

### Double-Blind Dose-Escalation Period

During the double-blind dose-escalation period, participants begin treatment with blinded study drug. The dose-escalation period will consist of the following weekly steps, based on the participant's body weight:
Dose Level 1:300 μg/kg (or 15 mg) maralixibat or placebo QD for 1 week
Dose Level 2:300 μg/kg (or 15 mg) maralixibat or placebo BID for 1 week
Dose Level 3:600 μg/kg (or 30 mg) maralixibat or placebo BID for the remaining duration of the study period

Dose escalation should occur in the absence of major safety (e.g., liver parameters) or tolerability (e.g., GI-related TEAEs) concerns related or possibly related to study drug. Investigators have until Week 4 to determine the maximum tolerated dose or reach Dose Level 3. The participant will remain on the maximum tolerated dose level (the dose the participant can administer without having safety or tolerability events related or possibly related to study drug) during the dose-escalation period for the remainder of the double-blind study period. The minimum dose to continue in the study will be 300 μg/kg (or 15 mg) QD.

Investigators should also review the study drug compliance during the dose-escalation period to ensure that the participant had adequate exposure to study drug to assess safety and tolerability. Any compliance concerns should be discussed with the medical monitor. Participants who cannot tolerate the minimum dose will be discontinued from the study.

Participants will undergo efficacy and safety assessments and procedures as specified in the SoA.

### Double-Blind Stable-Dosing Period

During the double-blind stable-dosing period, participants will be treated with 600 μg/kg BID or the maximum tolerated dose (determined during the double-blind dose-escalation period) of maralixibat or placebo.

Throughout the study, dose reductions to 300 μg/kg BID or 300 μg/kg QD for participants with body weight <50 kg or 15 mg BID or 15 mg QD for participants with body weight ≥50 kg or treatment interruptions are allowed for safety or tolerability reasons. After a dose interruption, participants should reinitiate dosing at their maximum tolerated dose, after agreement with the medical monitor. Participants will undergo efficacy and safety assessments and procedures as specified in the SoA.

### Open-Label Dose-Escalation Period

Participants who complete the double-blind period of the study and continue in the open-label period will receive maralixibat regardless of whether they were assigned to the maralixibat group or the placebo group in the double-blind period of the study. During the open-label dose-escalation period, all participants will receive maralixibat treatment. All participants will undergo dose escalation in order to maintain the blind. Investigators have until Week 24 to determine the maximum tolerated dose (the dose the participant can receive without having safety or tolerability events related or possibly related to study drug) for the open-label study period. The participant will remain on the maximum tolerated dose level for the remainder of the study. The minimum dose to continue in the study will be 300 μg/kg maralixibat QD for participants with body weight <50 kg and 15 mg QD for participants with body weight ≥50 kg.

Participants will undergo efficacy and safety assessments and procedures as specified in the SoA.

### Open-Label Stable-Dosing Period

During the open-label stable-dosing period, participants will be treated with 600 μg/kg BID or the maximum tolerated dose (determined during the open-label dose-escalation period) of maralixibat.

Participants will continue study treatment and will be monitored for safety and efficacy at every scheduled visit as described in the SoA.

Participants will remain in the study until 1) they are eligible to enter an alternative maralixibat study or expanded-access program, 2) maralixibat is commercially available, or 3) the sponsor stops the program.

### Safety Follow-Up Period

Participants will have a final safety follow-up contact 1 week after the administration of the last dose of study drug.

### Participant Completion and End of Study Definition

A participant is considered to have completed the study if the Week 20 visit has been completed (study completion). Participants may continue to receive maralixibat in the open-label extension period until study termination by the sponsor or transition to commercial product or alternative supply (e.g., expanded-access programs) of maralixibat.

The study termination date is defined as the date the final participant, across all sites, completes his or her final protocol-defined assessment. Note that this includes the safety follow-up contact.

Participants who discontinue at any time during this study will have ET assessments and a safety follow-up phone call, 7 days after ET visit. Participants who discontinue from the study for safety reasons will be followed as long as clinically indicated or until the safety finding is considered ongoing, stable, or resolved.

### Example 2: Study Population and Dosing

### Inclusion Criteria

Those who meet all of the following criteria during screening are eligible to participate in the study:
1. Informed consent and assent (as applicable)
2. Age ≥6 months at time of baseline visit
3. Diagnosis of cholestatic liver disease with cholestatic pruritus based on the following:
a. Chronic liver biochemical abnormalities (>90 days) and/or pathological evidence of progressive liver disease. Total sBA >2×ULN is required.
b. Persistent pruritus (>90 days). An average worst daily (morning and evening) ItchRO(Obs)/ItchRO(Pt) score ≥1.5 during the 2 consecutive weeks of the screening period leading to the baseline visit. If both instruments are administered, a score ≥1.5 is required only for ItchRO(Obs). Participants with the following diseases will be enrolled in the study: Any liver disease, including but not limited to the following: Alpha-1 antitrypsin deficiency, ARC syndrome, BA, Caroli's disease, ciliopathies, chronic viral hepatitis, hepatic sarcoidosis, idiopathic amyloidosis, IgG4-related sclerosing cholangitis, ischemic cholangiopathy, metabolic disorders, nonalcoholic fatty liver disease, post-liver transplant cholestasis (including patients with ALGS, PBC, PFIC, or PSC who have had liver transplant), secondary sclerosing cholangitis.
4. Completion of at least 10 valid daily (morning and evening) ItchRO(Obs)/ItchRO(Pt) entries during 2 consecutive weeks of the screening period, leading to the baseline visit. If both instruments are administered, the completion criterion is required only for ItchRO(Obs).
5. If taking antipruritics or ursodeoxycholic acid, the participant has to be on a stable dosing regimen (i.e., same dose and frequency in the 30 days prior to the screening visit and will continue this dosing regimen up to Week 40 [adjustment for body weight is allowed]).
6. Non-pregnant, non-lactating females of childbearing potential who are sexually active must agree to use at least an acceptable method of contraception during the study and for 30 days following the last dose of the study drug. Females of childbearing potential must have a negative pregnancy test result.
7. Access to email or telephone for scheduled participant contacts and access to smart phone or tablet for PROs
8. Ability to read and/or understand the questionnaires (both caregivers and participants ≥9 years of age)
9. For participants≤18 years of age: Access to consistent caregiver(s) during the study
10. Willingness (participant or caregiver) to comply with all study visits and requirements through the end of the study

### Exclusion Criteria

Those who meet any of the following criteria are NOT eligible to participate in the study:
1. Diagnosis of ALGS, ICP, PBC, PFIC, or PSC with native liver.
2. Current or recent history (<1 year) of atopic dermatitis or other non-cholestatic diseases associated with pruritus
3. History of decompensated cirrhosis or complications of cirrhosis (e.g., esophageal/gastric varices, ascites, hepatic encephalopathy, hepatorenal syndrome). In patients who have had a liver transplant, this exclusion criterion applies to the post-transplant period only. Patients with compensated cirrhosis with preserved hepatic synthetic function and absence of complications are eligible.
4. Suspected or proven cholangiocarcinoma or hepatocellular carcinoma
5. Unstable and/or serious medical disease that is likely to impair the ability to participate in all aspects of the study, confound efficacy and/or safety assessments, or result in substantially shortened life expectancy (e.g., any active malignancy including hematological malignancy, end-stage heart failure, active infection, acute and chronic diarrhea). Exceptionally, previous history of malignancy, adequately treated/in remission, that in opinion of investigator and medical monitor does not impact participant safety and participation in the study, may be allowed. The investigator should contact the medical monitor to discuss these cases and seek approval before the screening period.
6. Laboratory results during the screening visit as follows:

$$Platelet count \leq 150 , 000 / mm 3 \qquad a )$$

$$Albumin < 30 g / L \qquad b )$$

$$INR \geq \text{} 1.5 ( after intravenous or subcutaneous supplementation of vitamin K ) \qquad c )$$

$$Total bilirubin > 10 mg / dL \qquad d )$$

$$ALT > 10 \times ULN \qquad e )$$

7. Presence of any other disease or condition known to interfere with the absorption, distribution, metabolism, or excretion of drugs, including bile salt metabolism in the intestine (e.g., inflammatory bowel disease), per investigator discretion
8. Use of an IBAT inhibitor within 8 weeks prior to the screening visit
9. Use of any other investigational medication within 30 days or 5 times the half-life, whichever is greater, prior to the screening visit
10. Pregnant or nursing
11. Known intolerance/hypersensitivity to maralixibat or its excipients
12. History of nonadherence to medical regimens, unreliability, medical condition, mental instability, or cognitive impairment that, in the opinion of the investigator, could compromise the validity of informed consent, compromise the safety of the participant, or lead to nonadherence with the study protocol or inability to conduct the study procedures
13. Clinically relevant alcohol use disorder or drug abuse within 12 weeks of screening:
a. Moderate alcohol consumption as defined for this study by >1 and >2 standard drinks on average per day for women and men, respectively, within 12 weeks prior to the screening visit. A standard drink is defined as 44 mL (1.5 oz, one shot) of liquor, 148 mL (5 oz) of nonfortified wine, or 355 mL (12 oz) of beer (1 oz=29.57 mL)
b. Drug abuse within the 12 weeks prior to, or a positive drug screening result, at the screening visit unless it can be explained by a drug prescription. A positive drug screen will exclude participants if the investigator deems the result to be reflective of a pattern of behavior that might impair the participant's ability to participate in the study.
c. Use of cannabinoids (legal, prescribed, or otherwise) is allowed, provided use is stable (including as-needed use without need for increased frequency of use) for ≥12 weeks prior to the screening visit and throughout the entire study duration.

There are no diet or activity restrictions for this study.

Maralixibat chloride will be provided as an oral solution (20 mg/mL) along with 0.5-, 1.0-, and 3.0-mL sized dosing dispensers.

Participants <50 kg of body weight will receive maralixibat based on their individual body weight, up to 600 μg/kg BID.

Participants ≥50 kg of body weight will receive maralixibat up to 30 mg (1.5 mL) BID.

### Example 3: Efficacy Assessment

PRO questionnaires should consistently be given to participants or caregivers to complete before other assessments are done to minimize the potential impact a participant's discomfort could have on his or her answers.

### Itch-Reported Outcome (ItchRO)

Pruritus severity will be assessed using the Itch-observer/patient-reported outcome measure (ItchRO) Item 1 administered twice daily as described herein.

Caregivers for all participants≤18 years of age at screening will complete the Observer instrument (ItchRO [Obs]). The ItchRO(Obs) should be completed by the same caregiver for consistency, whenever possible.

Participants ≥9 years of age at screening will complete the patient instrument (ItchRO [Pt]). The same instrument assigned at study entry will be used throughout the study.

Participants and caregivers will be trained on the use of the data collection tool during the screening visit. Pruritus will be assessed and recorded twice daily, via ItchRO, beginning with the day after the screening visit and throughout the duration of the study.

Exceptionally, a paper version may be made available to accommodate cultural restrictions for a limited number of participants/families to complete the required assessments. However, any requests for paper version will need to be proactively approved by the sponsor, who would provide any officially licensed instruments for data collection.

The ItchRO(Obs) and ItchRO(Pt) eligibility (as applicable based on participant age) must be determined after a minimum of 2 weeks of twice-daily diary entry.

Postbaseline ItchRO(Obs)/ItchRO(Pt) results will remain blinded to sites and to the blinded study team until study unblinding (see Section 6.5).

ItchRO has been validated for patients with other cholestatic liver diseases (ALGS and PFIC) and will be validated for the study population with the use of PIC/CIC and PIS/CIS scores as anchors to help assess meaningful change for the ItchRO(Obs)/ItchRO(Pt) in the study.

### ItchRO(Obs)

The severity of pruritus will be measured by the completion of Item 1 in the ItchRO(Obs) (how severe were your child's itch-related symptoms). Caregivers will rate the severity of pruritus using 5 choices to describe the participant's itching condition. The sixth choice, “I don't know” will not count toward the severity score (categorized as missing data) and is included to account for rare occasions that the designated caregiver cannot observe the child, or the child could not communicate the severity of their itching condition. Capturing “I don't know” should be kept at an absolute minimum, as this will lead to missing data.

### ItchRO(Pt)

The severity of pruritus will be measured by the completion of Item 1 in the ItchRO(Pt) (how itchy did you feel). Participants will rate the severity of pruritus using 5 choices to describe their itching condition.

### Clinician Scratch Scale

The CSS provides an assessment of itch severity (see Appendix 5). The clinician's assessment of the participant's pruritus will focus on scratching and visible damage to the skin as a result of scratching as observed by the physician. The CSS uses a 5-point scale, in which 0 designates no evidence of scratching and 4 designates cutaneous mutilation with bleeding, hemorrhage, and scarring. A clinician's assessment of pruritus made by the principal investigator or subinvestigator or trained nurse using the CSS will be recorded at screening, baseline, and at additional study visits. To the extent possible, assessments should be made by the same individual during study visits.

### Patient Impression of Severity of Pruritus

The PIS is a questionnaire that will be administered to participants. Participants aged ≥9 years at screening will self-complete the questionnaire. The PIS is designed to assess the participant's perception of their itch severity. The questionnaire will be administered with a recall period of 1 week.

### Caregiver Impression of Severity of Pruritus

The CIS is a questionnaire that will be administered to caregivers of participants ≤18 years of age at screening. The CIS is designed to assess the caregiver's perception of itch severity of the child. The questionnaire will be administered with a recall period of 1 week.

### Patient Impression of Change

The PIC is designed to assess the participant's perception of his/her itching compared with his/her itching prior to the start of treatment with study drug. The PIC will be completed by participants who are ≥9 years of age at screening.

### Caregiver Impression of Change

The CIC is designed to assess the caregiver's perception of the participant's itch-related symptoms compared with his/her itch-related symptoms prior to the start of treatment with study drug. The CIC will be completed by all caregivers of participants≤18 years of age at screening.

### Liver Elastography

If participants undergo liver elastography as part of standard of care, the results will be entered into the eCRF, as indicated in the SoA.

### REFERENCES

Abetz-Webb L, Kennedy C, Hepburn B, et al. The burden of pruritus on patients with Alagille syndrome: results from a qualitative study with pediatric patients and their caregivers. Hepatology 2014; 60:526A-7A.
Baker A, Kerkar N, Todorova L, et al. Systematic review of progressive familial intrahepatic cholestasis. Clin Res Hepatol Gasteoenterol 2019; 43:20-36.
Beuers U, Wolters F, Oude Elferink R P J. Mechanisms of pruritus in cholestasis: understanding and treating the itch. Nat Rev Gastroenterol Hepatol 2023; 20:26-36.
Cheung, AC, Patel, H, Meza-Cardona, J, et al. Factors that influence health-related quality of life in patients with primary sclerosing cholangitis. Dig Dis Sci 2016; 61:1692-99.
Davit-Spraul A, Gonzales E, Baussan C, et al. Progressive familial intrahepatic cholestasis. Orphanet J Rare Dis 2009; 4:1.
Elisofon S, Emerick K, Sinacore J, et al. Health status of patients with Alagille syndrome. J Pediatr Gastroenterol Nutr 2010; 51:759-65.
European Commission. Ethical Considerations for Clinical Trials on Medicinal Products Conducted with Minors. 2017. Available online at https://health.ec.europa.eu/system/files/2018-02/2017_09_18_ethical_consid_ct_with_minors_0.pdf. [Accessed 26 Feb. 2024].
[FDA] US Food and Drug Administration. Decentralized clinical trials for drugs, biological products, and devices. Draft Guidance May 2023. Available online at https://www.fda.gov/media/167696/download?utm_medium=email&utm_source=govdelivery [Accessed 27 Feb. 2024].
[FDA] US Food and Drug Administration. Patient-focused drug development: selecting, developing, or modifying fit-for-purpose clinical outcome assessments. Draft Guidance June 2022. Available online at https://www.fda.gov/regulatory-information/search-fdaguidancedocuments/patient-focused-drug-development-selecting-developing-or-modifying-fitpurpose-clinical-outcome [Accessed 27 Feb. 2024].
Haapamaki J, Tenca A, Sintonen H., et al. Health-related quality of life among patients with primary sclerosing cholangitis. Liver Int 2015; 35:2194-201.
Hirschfield G M. Diagnosis of primary biliary cirrhosis. Best Pract Res Clin Gastroenterol 2011; 25:701-12.
Hounnou G, Destrieux C, Desmé J, et al. Anatomical study of the length of the human intestine. Surg Radiol Anat 2002; 24:290-4.
Kamath B M, Chen Z, Romero R, et al. Quality of life and its determinants in a multicenter cohort of children with Alagille syndrome. J Pediatr 2015; 167:390-6.e3.
Kamath B M, Baker A, Houwen R, et al. Systematic review: the epidemiology, natural history, and burden of Alagille syndrome. J Pediatr Gastroenterol Nutr 2018; 67:148-56.
Kremer A E, Oude Elferink R P, Beuers U. Pathophysiology and current management of pruritus in liver disease. Clin Res Hepatol Gastroenterol 2011; 35:89-97.
Kronsten V, Fitzpatrick E., Baker. Management of cholestatic pruritus in paediatric patients with Alagille syndrome: the King's College Hospital experience. J Pediatr Gastroenterol Nutr 2013; 57:149-54.
Kuo A, Gomel R, Safer R, et al. Characteristics and outcomes reported by patients with primary sclerosing cholangitis through an online registry. Clin Gastroenterol Hepatol 2019; 17:1372-8.
Lee W S, Chai P F, Looi L M. Progressive familial intrahepatic cholestasis in Malaysian patients—a report of five cases. Med J Malaysia 2009; 64:216-9.
Lindor K D, Gershwin M E, Poupon R, et al. Primary biliary cirrhosis. Hepatology 2009; 50:291-308.
Lu M, Zhou Y, Haller I V, et al. Fibrotic liver disease consortium investigators. Increasing prevalence of primary biliary cholangitis and reduced mortality with treatment. Clin Gastroenterol Hepatol 2018; 16:1342-50.
[NIAAA] National Institute on Alcohol Abuse and Alcoholism. Available at https://www.niaaa.nih.gov/what-standard-drink. [Accessed 27 Feb. 2024].
Patel S P, Vasavda C, Ho B, et al. Cholestatic pruritus: Emerging mechanisms and therapeutics. J Am Acad Dermatol 2019; 81:1371-8.
PSC Support. Clinical need in PSC and clinically meaningful change: what is important to patients? 2016. Available at: https://www.pscsupport.org.uk/wpcontent/uploads/2019/06/PSC-Support-Patient-Survey-Results.pdf [accessed 27 Feb. 2024].
Raszeja-Wyszomirska J, Wunsch E., Krawczyk M, et al. Prospective evaluation of PBC-specific health-related quality of life questionnaires in patients with primary sclerosing cholangitis. Liver Int 2015; 35:1764-71.
Shneider B L, Mazariegos G V. Biliary atresia: a transplant perspective. Liver Transpl 2007; 13:1482-95.
Sundaram S S, Mack C L, Feldman A G, et al. Biliary atresia: Indications and timing of liver transplantation and optimization of pretransplant care. Liver Transpl 2017; 23:96-109.
Thébaut A, Debray D, Gonzales E. An update on the physiopathology and therapeutic management of cholestatic pruritus in children. Clin Res Hepatol Gastroenterol 2018; 42:103-9.
Vandriel S M, Li L T, She H, et al.; The GALA Study Group. Natural history of liver disease in a large international cohort of children with Alagille syndrome: results from The GALA Study. Hepatology 2022; 77:512-29.
Whitington P F, Freese D K, Alonso E M, et al. Clinical and biochemical findings in progressive familial intrahepatic cholestasis. J Pediatr Gastroenterol Nutr 1994; 18:134-41.
Whitington P F, Whitington G L. Partial external diversion of bile for the treatment of intractable pruritis associated with intrahepatic cholestasis. Gastroenterology 1988; 95:130-6.
Yu H, Wangensteen K, Deng T, et al. MRGPRX4 in cholestatic pruritus. Semin Liver Dis 2021; 41:358-67.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1. A method for treating a rare cholestatic liver disease in a subject in need thereof comprising administering to the subject about 300 μg/kg/day to about 1200 μg/kg/day maralixibat, or a pharmaceutically acceptable salt thereof.

2. A method of reducing cholestatic pruritus in a subject having a rare cholestatic liver disease comprising administering to the subject about 300 μg/kg/day to about 1200 μg/kg/day maralixibat, or a pharmaceutically acceptable salt thereof.

3. The method of claim 1, wherein the rare cholestatic liver disease is selected from the group consisting of Caroli disease, Caroli syndrome, ciliopathies (Joubert syndrome, Meckel-Gruber syndrome, NPHP3 mutation), alpha-1-antitrypsin deficiency, chronic idiopathic hepatitis, secondary sclerosing cholangitis related to COVID-19, secondary sclerosing cholangitis related to injury, IgG4-related sclerosing cholangitis, ARC, Langerhans cell histiocytosis, sodium taurocholate co-transporting polypeptide deficiency, transaldolase deficiency, X-linked myotubular myopathy, hepatic sarcoidosis, idiopathic amyloidosis, ischemic cholangiopathy, rare metabolic disorders, post-liver transplant cholestasis, and undiagnosed cholestatic pruritus.

4. (canceled)

5. The method of claim 1, wherein the rare cholestatic liver disease is biliary atresia (BA).

6.-8. (canceled)

15. The method of claim 1, wherein the treatment comprises reducing one or more of cholestatic pruritus, serum bile acids, and bilirubin.

16. (canceled)

17. (canceled)

18. The method of claim 1, wherein the pharmaceutically acceptable salt of maralixibat is maralixibat chloride.

19. The method of claim 1, wherein the maralixibat or pharmaceutically acceptable salt thereof is administered in an amount from about 400 μg/kg/day to about 1200 μg/kg/day.

20. The method of claim 1, wherein the maralixibat or pharmaceutically acceptable salt thereof is administered in an amount from about 600 μg/kg/day to about 1200 μg/kg/day.

21. The method of claim 1, wherein the maralixibat or pharmaceutically acceptable salt thereof is maralixibat chloride, and maralixibat chloride is administered in an amount of about 1200 μg/kg/day.

22. The method of claim 1, wherein the maralixibat or pharmaceutically acceptable salt thereof is administered in an amount of about 0.5 mg/day to about 100 mg/day.

23. The method of claim 1, wherein the maralixibat or pharmaceutically acceptable salt thereof is administered in an amount of about 10 mg/day to about 60 mg/day.

24. The method of claim 1, wherein the subject has intermittent cholestasis.

25. The method of claim 1, wherein the subject has undergone biliary diversion surgery or liver transplantation.

26. (canceled)

27. The method of claim 1, wherein the subject is a pediatric subject.

28. (canceled)

29. (canceled)

30. The method of claim 1, wherein the subject is an adult.

31. The method of claim 1, wherein the maralixibat or pharmaceutically acceptable salt thereof is administered once daily (QD).

32. The method of claim 1, wherein the maralixibat or pharmaceutically acceptable salt thereof is administered twice daily (BID).

33. The method of claim 1, wherein the maralixibat or pharmaceutically acceptable salt thereof is maralixibat chloride, and maralixibat chloride is administered at 600 μg/kg/day BID for a total daily dose of 1200 μg/kg/day.

34. The method of claim 2, wherein the reduction of cholestatic pruritus is a reduction of an ItchRO score, of an ItchRO(Obs) score, of a CSS score, of a patient impression of severity of pruritus (PIS), of a caregiver impression of severity of pruritus (CIS), of a patient impression of change (PIC), or of a caregiver impression of change (CIC), or a combination thereof.

35. The method of claim 1, wherein the administration of the maralixibat or pharmaceutically acceptable salt thereof reduces an ItchRO(Obs) score of the subject by at least 1.0 points, or at least 1.2 points, or at least 1.4 points, or at least 1.6 points relative to baseline.

36.-38. (canceled)

39. The method of claim 1, wherein the administration of the maralixibat or pharmaceutically acceptable salt thereof reduces a CSS score of the subject by at least 1.0 points, or at least 1.2 points, or at least 1.4 points, or at least 1.6 points relative to baseline.

40.-42. (canceled)

43. The method of claim 1, wherein the administration of the maralixibat or pharmaceutically acceptable salt thereof reduces sBA concentration in the subject by at least 50 μmol/L relative to baseline.

44. The method of claim 1, wherein the administration of the maralixibat or pharmaceutically acceptable salt thereof reduces total bilirubin by at least 0.2 mg/dL relative to baseline.

45. The method of claim 1, further comprising administering a lipid soluble vitamin (LSV) in subjects with LSV deficiency.

46. (canceled)

47. The method of claim 1, wherein the maralixibat or pharmaceutically acceptable salt thereof is administered before a meal.

48. The method of claim 47, wherein the maralixibat or pharmaceutically acceptable salt thereof is administered about 30 minutes before a meal.

49. The method of claim 47, wherein the maralixibat or pharmaceutically acceptable salt thereof is administered BID, about 30 minutes before the morning meal and about 30 minutes before the evening meal.

50. The method of claim 1, wherein the maralixibat is administered in the form of a pharmaceutical composition comprising maralixibat, or a pharmaceutically acceptable salt thereof, an antioxidant, and a preservative.

51. The method of claim 50, wherein the pharmaceutical composition is a liquid composition for oral administration.

52.-55. (canceled)

56. The method of claim 50, wherein the pharmaceutical composition is a solid composition for oral administration.
